(19) United States
(12) Patent Application Publication
(10) Pub. No.: US 2026/0248726 A1
(43) Pub. Date: Aug. 27, 2026

(54) MONODISPERSE NANOFIBROUS MICROPARTICLES FOR DELIVERY OF ONE OR MORE ACTIVE SUBSTANCES

(71) Applicant: Danmarks Tekniske Universitet, Kongens Lyngby (DK)

(72) Inventors: Lasse Højlund Eklund Thamdrup, Kongens Lyngby (DK); Anja Boisen, Kongens Lyngby (DK); Fatemeh Ajalloueian, Kongens Lyngby (DK)

(21) Appl. No.: 19/161,072

(22) PCT Filed: Feb. 29, 2024
(86) PCT No.: PCT/EP2024/055263

§ 371 Date: Aug. 29, 2025

(30) Foreign Application Priority Data
Mar. 3, 2023 (EP) 23159954.9

(51) Int. Cl. A61K 9/16; A61K 31/43

(52) U.S. Cl. CPC A61K 9/1694; A61K 9/1647; A61K 9/1652; A61K 31/43

(57) ABSTRACT

Disclosed herein are a new type of advanced microparticles for delivery of an active substance. The microparticles exhibit a complex and porous bulk morphology composed of one or more layers of electrospun nanofibrous materials and/or compounds.

### FIELD OF THE INVENTION

The invention relates to monodisperse microparticles with a complex inner morphology comprising one or more consecutive layers of electrospun nanofibers. The microparticles can exhibit a tuneable compactness/porosity and they are targeting delivery of active substances such as, e.g., drug substances, vaccines, microorganisms (e.g. probiotics), vitamins etc. The microparticles are prepared by cutting an electrospun sheet with specific thickness using an ultrasharp micro-cutting tool. One or more biocompatible and/or biodegradable polymers or other substances capable of being electrospun and optionally other ingredients such as one or more active substances are electrospun to form a nanofibrous sheet. Cutting the electrospun sheet results in nanofibrous monodisperse microparticles having a well-defined shape and having a very narrow particle size distribution and a well-defined topography. Said topography and size are contemplated to be beneficial in terms of enhancing properties (e.g., mucoadhesion, fast or sustained released) and/or ensuring compatibility with a desired administration route (i.e., oral, nasal, pulmonary etc.) in those cases where the active substance is a drug substance and the microparticles are part of a delivery system that is a drug delivery system. The electrospun sheet may contain more than one layer and each layer may be the same or different from the other layers e.g. regarding nature and concentration of polymer(s), active substance(s), excipient(s) etc. Moreover, the individual layer(s) may have the same or different thickness.

### BACKGROUND OF THE INVENTION

Drug delivery systems have been developed since the beginning of the 1970'ties, and many different delivery systems have been reported for various administration routes.

Oral drug delivery has been recognized as one of the most attractive methods among various delivery routes. Oral dosage forms are still the gold standard for the treatment and management of chronic and debilitating diseases, such as cancer, various infections, neurodegenerative and psychotropic disorders. Ease of administration, potential for applying solid formulations providing sustained delivery and long shelf life are key reasons for the significant tendency towards use of oral delivery systems. Especially, several pharmaceutical investigations have focused on developing oral dosage forms for tunable sustained drug release. Conventional dosage forms for oral delivery are tablets, powders, films, or liquids. Compared to conventional single-unit dosage forms, micro- and nanoparticles have gained increasing interest for development of novel gastrointestinal drug delivery systems.

Micro- or nanoparticulate dosage forms have appeared promising due to their small size where a number of separate subunits (instead of a single-unit dosage form) are administered per each dose of drug. It is particularly important since use of commercial single-unit dosage forms is associated with challenges such as unpredictable disintegration, nonspecific drug release, or dose dumping.

Comparing microparticles and nanoparticles, some superiorities are reported for nanoparticles. The larger surface-area-to-volume ratio of nanoparticles can increase the interaction with the mucosal surface and the solubilization of drugs, and may lead to more uniform distribution and elevated drug release, as well as higher cellular uptake. There are, however, some drawbacks associated with the use of nanoparticles for oral drug delivery. Premature drug leakage is a major drawback with use of nanoparticles for applications like colon-targeted drug delivery. On the other side, for applications like peptide or insulin delivery, microparticle drug delivery systems have demonstrated advantages over nanoparticle systems, where microparticulate formulations can enhance peptide stability, improve protection against enzymatic degradation, and facilitate oral absorption by paracellular, transcellular and lymphatic routes. Such dual characteristics raise the need to make use of both nano- and microparticulate formulations for maximum benefit from both structures. However, it has been a long-lasting challenge realizing nano-structured microparticles for biomedical applications.

Independently of the fabrication technology that has been applied, the currently available processes have been unable to provide i) uniform particle size and shape and ii) tuneable multi drug content and release. The nano-base in these so-called nanostructured microparticles has either been nanoparticles (NPs) or nanofibers (NFs). For NPs, an example is the work by Augustine et al where they developed a nanoparticle-in-microparticle system for enhanced delivery of hydrophobic drugs in general, along with providing further protection for passage through the stomach. They demonstrated the potential of this structure to treat HIV, where a significantly increased oral bioavailability of antiretrovirals (ARVs) was observed¹. In such systems, the level of porosity is usually low and there is limited control over distribution of NPs inside the microparticles^{2,3}. Regarding NFs, the application of NFs for drug delivery has mostly been limited to electrospun nanofibrous sheets that are used as dressings. There have also been a few cases where NFs are used to make microparticulate formulations. In these works, the electrospun NFs have been chopped into individual fibrous segments and further mixed with organic solutions to undergo complex multi-step chemical procedures forming particulate forms^{4,5}. Due to several wet-chemical procedures applied post-electrospinning and the risk of premature drug leakage, these nanostructured microparticles are not appropriate for drug delivery, but have shown promising results for cell-delivery applications^{4,5}. Furthermore, the original deeply interconnected web of NFs has been ruined after chopping the NFs. To address this issue, Park and colleagues performed freeze-milling of the original web of nanofibers, followed by suspension and filtration to collect the nanofibrous microparticles.

The issue with this method and another study applying electrospraying to form nanofibrous microspheres^{6,7} is the limited control over the final shape, compactness level and the size of the particles⁷.

Microparticles have been Subject of a Number of Patents and Publications:

US 2015/133454 (Choy et al.) relates to nanostructured mucoadhesive microparticles. The microparticles have a surface with a nanostructure form, thereby having an enlarged surface area and an increased adhesiveness to a mucous membrane. The nanostructures may be prepared by electrospinning. The nanostructured microparticles are prepared by electrospinning to give a nanofibrous sheet, which then is freeze-milled by a steel impactor. The microparticles may be suspended in a liquid and filtered to obtain a particle size below 10 microns, however the microparticles obtained seem to have a relatively wide particle size distribution.

WO 2018/212758 relates to a carrier system for biomaterials and biomolecules. In particular, it describes a method of preparing platelet-rich fibrin (PRF) as a drug delivery system. It also describes a composition comprising PRF and a regenerative biomaterial. PRF may also be combined with porous injectable particles, polymers etc. and the porous particles seem to be prepared by electrospinning. There are no details regarding the electrospinning process and there are no details regarding the size of the porous particles.

Indian patent application 201841042298 relates to platelet-rich plasma (PLP) based formulations for treating and managing wounds. PLP may be encapsulated in nano- or micromaterials. A nonfibrous patch may be formed by electrospraying the PLP and a polymer. Also described is the preparation of microparticles by electrospraying a core solution and a shell polymer solution to obtain core-shell microparticles that will encapsulate PLP.

WO 2014/058342 (000 Stalvek) relates to biophosphate ceramic materials for medicine, more specifically for traumatology, orthopedics, reconstructive surgery, cosmetology, and dentistry, and to a drug delivery system. The porous microspheres are granules based on calcium phosphate, obtained from biological hydroxyapatite, and magnesium orthophosphate. The mixture from which the granules are formed presumably by electrospraying also contains 1-3 percent sodium alginate solution in distilled water, and a curing agent, namely a saturated solution of calcium chloride. The granules are heat treated. The granules have a size within the range of from 10 μm to 1000 μm and exhibit biocompatibility, biodegradability and osteoinductive and osteoconductive properties.

CN 103585635 (Univ Jiangsu) discloses a slow-release polylactic acid microsphere capable of maintaining protein and polypeptide drug activity and a preparation method thereof. The slow-release polylactic acid microsphere comprises 5-30 parts of a protein and polypeptide drug, 30-90 parts of a polylactic acid biodegradable material and 5-50 parts of nano-bioactive glass. The slow-release polylactic acid microsphere seems to be prepared by an electrospraying technique, and mainly comprises the polylactic acid biodegradable material (PLGA (polylactic acid-glycolic acid copolymer) or PLA (polylactic acid)), the bioactive glass and a main drug. The slow-release polylactic acid microsphere is said to greatly promote activity retention of the protein and polypeptide drug in a preparation process, and can improve stability of the protein and polypeptide drug in microsphere storage and release processes.

US 2008/0131509 (Hossainy et al.) relates to a composition comprising a silk protein. The composition may comprise a carrier. Biodegradable carriers in the form of electrospun nanofibers or microfibers are described, and microparticles are described. The microparticles may be prepared by electrospraying.

Boda et al. (ACS Appl Mater Interfaces, 2018 Aug. 1; 10(30)m 25069-25079) relates to microspheres prepared by electrospraying a dispersion of electrospun nanofiber segments. A method is described, where fibers are obtained by electrospinning, these fibers are cut into segments using cryocoolant and then the fiber segments are dispersed in a medium that is subject to electrospraying.

CN 108078954 (Univ Jilin) relates to preparation of drug-carrier microspheres, and discloses an electrospraying method for coaxially preparing an injectable PLGA (poly(lactic-co-glycolic acid)) drug-carrier microsphere. The coaxial drug-carrier microspheres prepared by the invention can be subjected to sustained-release and controlled-release and can be used for injecting.

Although microparticles have been subject of many publications, there is still a need for developing mono- or multilayer microparticles of a well-defined nanostructure, a well-defined shape, a scalable compactness (porosity) level, and a well-defined mean particle size with a narrow size distribution, i.e. there is a need for developing techniques for preparing monodisperse nanostructured microparticles for controlled multi-drug delivery. Besides, there is a need to apply more green and energy saving technologies where usage of organic solvents is minimized and the whole procedure is shortened and simplified.

### SUMMARY OF THE INVENTION

In its broadest aspect, the present invention relates to monodisperse nanostructured microparticles with uniform shape and compactness level, wherein the microparticles comprise electrospun fibers (FIG. 1). The fibers have a diameter of nano size, i.e. the microparticles comprise electrospun nanofibers. The microparticles are obtained by electrospinning a liquid comprising one or more polymer and, optionally, one or more active substance(s), whereby a nanofibrous sheet (with tunable thickness) of one or more polymer(s) and the optional active substance(s) is obtained, and the sheet is then cut into monodisperse microparticles of uniform shape. The microparticles may be in the form of mono-layered or multilayered fibers. The microparticles may contain one or more active substance(s) such as, e.g., one or more drug substance(s) per each electrospun sheet. If the microparticles contain multilayered fibers, the individual layers may be the same or different; thus, each layer may contain the same or a different polymer, the same or a different active substance, the same or different excipient(s) and/or the concentration of the ingredients in each layer may be the same or different. We can form core-shell-like microparticles if a three-layer electrospun sheet with two similar external layers are cut, and the level of core protection could be varied by altering the thickness of the corresponding external layers. Moreover, the thickness of each layer may be the same or different.

The microparticles may find use in various technical areas for delivering an active substance to a particular target. The active substance may be a pharmaceutically active substance, a physiologically active substance, a drug substance, a biologically active substance such as, e.g., a vitamin, a peptide, a co-factor, etc. or a biologically active substance for growth regulation of plants, and animals. The term “active substance” also includes bioactive agents such as DNA. RNA, bacteria, cells, etc. Herein the term “delivery system” is used in its broadest sense to denote a delivery system for an active substance. Specifically, e.g., when the term “drug delivery system” is used it denotes a delivery system for drug substances.

Compared with other technologies to provide microparticles, the key differences of the present microparticles are associated with the nanofibrous morphology obtained by electrospinning of individual layers that may form the final electrospun sheet. By using electrospinning to prepare the nanofibrous sheet and cutting the electrospun sheet subsequently, nanofibrous microparticles with different degrees of compactness and porosity obtained. The morphological properties of these microparticles differ from conventional bulk microparticles and patches, which are fabricated using other techniques. The nanofibrous microparticles of the present invention benefit from the increased surface area and porosity of the electrospun nanofibers, where the compactness level of the final particles is tunable depending on the original thickness (height) of the electrospun sheet compared with the depth (height) of the cutting tool. In addition, the dissolution behaviour is not only governed by erosion from exposed surfaces, but also from the polymers used and the porosity and/or permeability of the microparticles (especially with regard to the order of the layers if using multilayer electrospun sheets). The hydrophobicity can be tuned and by e.g. using a hydrophilic polymer the water uptake can be changed etc.

Depending on the 3D structure and the size of the cutting tool, monodisperse microparticles with potential sizes from 5 to 500 μm and size-deviation of 2.5-10% can be formed; where at least 90% of the particles have a size of from −10% of the mean particle size to +10% of the mean particle size, or such as from −5% to +5% or from −2.5% to 2.5% of the mean particle size.

In those cases, where the microparticles contain a drug substance and the drug delivery system is designed to be administered by oral, nasal, pulmonal, topic or parenteral administration, then the mean particle size is typically from 1 μm to 120 μm such as, e.g., from 1 μm to 20 μm (pulmonal administration) or from 30 μm to 120 μm (nasal administration). The mean particle size is less important for the oral or topic administration routes.

The uniform shape and size (i.e. monodispersity) of the microparticles is obtained using a microfabricated tool, i.e., “cutting tool”. The tool can either comprise i) close-packed or isolated compartments separated by protrusions featuring sharp cutting edges or ii) a grid of interconnected sidewalls, that are not terminated at the bottom, where the top edge of the sidewalls represents the sharp cutting edges of the tool (FIGS. 2, 4 and 5). As the tool is microfabricated using low tolerance processes, the cut microparticles will be similar in both shape and size and both are largely governed by the topography of the microfabricated cutting tool.

The invention also relates to compositions comprising such microparticles and for the use thereof e.g. in medicine.

The microparticles or the compositions comprising the microparticles may be used as delivery systems for one or more active substance(s), such as for one or more drug substance(s) (drug delivery system).

Described herein is also a method for preparing monodisperse microparticles, the method comprises the steps of
i) electrospinning a solution comprising one or more polymer(s) and, optionally, one or more active substance(s) (including drug substance(s), etc.), to obtain a fiber sheet comprising the one or more polymer(s) and the optional one or more active substance(s),
ii) cutting the fiber sheet obtained by a cutting tool to obtain monodisperse microparticles with uniform shape and size. (FIG. 3A).
iii) transfer step after cutting to collect particles (FIG. 3B).

More specifically, a method for preparing monodisperse microparticles comprises the steps of
i) electrospinning a solution comprising one or more polymer(s) to obtain a mono-layered nanofibrous sheet or electrospinning several solutions each containing of one or more polymer(s), one after another, to obtain a multi-layered nanofibrous sheet. The solution(s) may optionally contain a substance such as a drug substance, and each solution may be the same or different e.g., containing the same or different polymer(s) suitable for electrospinning and/or the same or different active substance(s), and, moreover, the concentration(s) of the polymer(s) and/or active substance(s) in each solution may be the same or different,
ii) potentially covering the electrospun sheet obtained on top and/or bottom side with a solid polymeric layer of polymer or metal nanoparticles to provide e.g. pH sensitive coatings or propellants e.g., for potential micromotor applications,
iii) cutting the nanofibrous sheet using a cutting tool to obtain microparticles with uniform shape and size.,
iv) A transfer step after cutting allows for easy harvesting of the microparticles which are residing inside the concave compartments of the micro-cutting tools

### DEFINITIONS AND DESCRIPTION OF TERMS USED HEREIN

Monodisperse—In the present disclosure, the term “monodisperse” refers to an ensemble of produced microparticles having a narrow size distribution. Essentially the particle distribution is characterized by a mean particle size and a relevant tolerance, which could be ±10% or ±5% or ±2.5%. When the monodisperse particles are used in pharmaceutical compositions, monodispersity is not a prerequisite for all administration routes. However, it may be essential for some administration routes (e.g., pulmonary) to ensure that the pharmaceutical composition is delivered to the target location.

Particle size: As described herein before, the particle size will depend on the use of the microparticles. In the case of microparticles for use as or in drug delivery systems, the particle size will depend on the desired route of administration. In general, a nanoparticle is a particle with a size of from 0.1 to 100 nm, a microparticle is a particle with a size of from 0.1 to 100 μm, and a milliparticle has a size of from 0.1 to 100 mm. However, in the present context, the term “microparticles” are used for particles having a mean particle size in the 1-1000 μm range.

Dealing with several administration routes where the particles may be in liquid and/or airborne, the particle size can be defined by the hydrodynamic diameter or radius. Said particle size can be quantified by making particle size analysis measurements on liquid dispersions or dry powders. The relevant range of particle sizes is currently believed to be 1-500 μm. This covers particles for more or less all administration routes covering (but not limited to) pulmonary, nasal, buccal, oral, vaginal, rectal, parenteral and ophthalmic.

In the present context, the term “monodisperse microparticle” is used for a microparticle having a size from 1 to 500 μm, and wherein the particle size distribution is narrow and at the most spans from −10% of the mean particle size to +10% of the mean particle size; or +5% or ±2.5% of the mean particle size, and wherein the particle size distribution and the mean particle size are determined by suitable methods including SEM imaging and particle size analysis. The term “monodisperse” relates to the spatial size of an ensemble of entities. More specifically, an ensemble of particles, grains etc. is said to be monodisperse if the particle size and/or the volume of the particles are similar. This can be measure of that the particle size distribution is narrow, which in turn means that the constituent entities have approximately the same size, or it can be measured by SEM or by volume. For example, the size of the monodisperse particles may span from −10% of the mean particle size to +10% of the mean particle size such as, e.g., ±5% or ±2.5% of the mean particle size. If the monodispersity is measured by SEM the particles defined by a specific parameter such as the length of the largest dimension, then the length may span from −10% of the mean particle size to +10% of the mean particle size such as, e.g., ±5% or ±2.5% of the mean particle size. If the monodispersity is measured by volume, the volume of the particles may span from −10% of the mean particle size to +10% of the mean particle size such as, e.g., ±5% or ±2.5% of the mean particle size. Monodispersity also includes that the particle have the same shape. Ths,monodisperse particles are of uniform size and shape. In the present context, the term “uniform size” refers to that each particle has almost the same size as the other particles ±10% or ±5% or ±2.5% as determined by particle size analysis or SEM imaging.

In the present context, the term “uniform shape” refers to that each particle has almost the same shape and 3D structure as the other particles and the volume of the particles differs with ±10% or ±5% or ±2.5%.

Electrospinning—Electrospinning is a term derived from “electrostatic spinning,” and defines an electrohydrodynamic fiber production method that uses electric fields to draw charged threads of polymer solutions (or polymer melts) forming a sheet of fibers with diameters in the order of some hundred nanometers (100 nm up to 10 μm). The electrospun nanofibers are characterized by a mean diameter in the range of 100-1000 nm. Especially for particles aimed at pulmonary delivery, the nanofiber diameter should be kept small (i.e., up to app. 1/10 the particle size).

In the present context, the term “active substance” refers to an active substance that may be a pharmacologically active substance, a physiologically active substance, a drug regulation agent of plants, animals or fish or a pesticide, insecticide, or fungicide etc. The term “active substance” also includes bioactive agents such as DNA. RNA, bacteria, cells, etc. The term “drug substance” is intended to denote a substance that is therapeutically active, a substance that is pharmacologically active, and/or a substance that is physiologically active.

Pharmaceutical composition—In the present disclosure, the term “pharmaceutical composition” refers to a composition intended for conferring a pharmacological (i.e. therapeutical) and/or physiological effect to an organism to which the pharmaceutical composition is applied. The effect is obtained by the action of a therapeutically or physiologically active compound contained in the composition. Pharmaceutical compositions cover (but may not be limited to) drug substances (including peptide drugs etc.), vaccines and probiotics.

Drug—The term ‘drug’ covers drug substances as well as formulations containing one or more drug substance such as pharmaceutical composition.

Drug delivery system—A drug delivery system (DDS) is a formulation or a device that delivers a drug substance to a specific site of a body. It may enable a drug substance to selectively reach its site of action e.g., by controlling the rate at which a drug is released and the location in the body where it is released. Herein the term “delivery system” is used in its broadest sense, i.e. it denotes a delivery system for an active agent.

(Electrospun) sheet—In the present disclosure, the term “sheet” refers to a material stack composed of one or more electrospun nanofibrous layers where the width and length of the material stack are orders of magnitude larger than the thickness of said stack. Depending on the target particle size requirements and depending on the depth of the cutting tool, the sheet may have an overall thickness ranging from 50-200 μm.

Cutting: In the present disclosure, the term “cutting” refers to the physical method by which the electrospun sheet is shaped into an ensemble of discrete microparticles. Cutting is conducted at room temperature by applying a force/pressure to a microfabricated tool featuring sharp cutting edges. The electrospun sheet, optionally consisting of multiple layers, is cut by shearing and breaking of the fibers along the cutting edges.

Cutting tool—In the present disclosure, the term “tool” refers to a microfabricated planar or cylindrical (most likely a planar tool adapted to a cylindrical roller) entity featuring discrete or interconnected cutting edges having a single apex with a width or tip radius not exceeding 2 μm. The cutting tool is micro-structured tool that is used for cutting through the electrospun sheet to make the nanofibrous microparticles. This allows for producing monodisperse particles comprising one or more (limit realistic interval—say 1-10) layers of electrospun polymers.

### DETAILED DESCRIPTION OF THE INVENTION

The invention relates to monodisperse microparticles with uniform shape, wherein the microparticles comprise electrospun fibers.

The electrospun fibers are obtained by electrospinning a solution comprising one or more polymer(s) to obtain a nanofibrous mono-layered sheet (FIG. 6) that is cut into monodisperse nanofibrous microparticles “MoNaMi”. Multi-layered sheets can be obtained by electrospinning two or more solutions each containing one or more polymer(s) (FIG. 7), and, optionally one or more active substance(s). The electrospun material from the two or more solutions is collected on the same collector, i.e. a nanofibrous multi-layered sheet is obtained and is then cut into monodisperse microparticles where the multilayered nanofibrous microparticles is called “MuNaMi”.

The electrospun monodisperse microparticles may contain an active substance. Typically, the active substance is incorporated into the electrospun nanofibrous material, i.e., the solution subjected to electrospinning also contains the active substance.

The electrospun monodisperse microparticles according to the invention may be used in different technical fields including medicine, agriculture, delivery of active substances, drug delivery systems etc. In the present context, focus is on the use of microparticles as drug delivery systems.

For drug delivery applications the microparticles may be administered e.g., by oral, nasal, topic, parenteral and pulmonary routes. The optimum particle size depends on the intended use. For instance, administration by the pulmonary route requires a smaller mean particle size than administration by the oral administration route.

Microparticulate dosage forms are advantageous compared with single-unit dosage forms, since challenges such as unpredictable disintegration, nonspecific drug release, or dose dumping which are associated with use of single-unit dosage forms are alleviated. Microparticles have a large surface-area-to-volume ratio, which can provide a greater surface area for interaction with the mucosal surface and for the solubilization of drugs leading to increased bioavailability. Other advantages include more uniform distribution and elevated drug release, and increased residence time of particles in the GI tract.

Pulmonary drug delivery is also an attractive approach thanks to the highly vascularized large surface area of the lung, and minimal metabolizing enzymes present in the lung. In contrast, oral drug delivery to or via the gastrointestinal tract leads to a pronounced contact with metabolizing enzymes present in the gastrointestinal tract. Moreover, pulmonary drug delivery reduces the systemic side effects as observed after intravenous administration. The current available therapies have showed poor bioavailability of drugs at the target site. However, advanced drug delivery systems including microparticles have gained attention for improving the therapeutic efficacy of poorly bioavailable drugs, prolonged and controlled drug release, and reduced off-target side effects.

Microparticle-based formulations can encapsulate nano-formulations and increase the stability of drugs and provide sustained release at the target site (deeper layers of lung). Microparticles have been found to show deposition in the deeper lung areas compared with nanoparticles. Especially large and porous particles (diameter more than 5 μm) are reported to be more effective compared with nonporous particles for pulmonary drug delivery due to low density and their better aerodynamic behaviour in airways. Also, large porous particles tend to disaggregate more easily under shear forces. Overall, the microparticles of the present invention fully meet such requirements thanks to their porous nano-structured microparticulate characteristics.

The invention is a platform technology and the microparticles may be prepared using a variety of polymers, a variety of solvents/dispersion media for the electrospinning process, a variety of drugs/bioactive agents, a variety of shapes, a variety of sizes etc.

### Monodisperse Microparticles

The invention provides monodisperse microparticles with uniform shape, wherein the microparticles comprise electrospun fibers.

Electrospun fibers are obtained by subjecting polymeric solutions or polymeric dispersions to electrospinning, i.e., to a method involving electro-hydrodynamic forces to obtain a sheet of fibers. In general, such methods are denoted electrohydrodynamic (EDH) methods and include electrospinning, electrospraying, coaxial electrospinning etc. In the present context, electrospraying methods are fundamentally similar to electrospinning. However, electrospraying works with low viscosity solutions or emulsions that lead to particles and not continuous fibers. Normally, electrospraying can be considered as a method to make particles of various sizes, since when using electrospraying there is no control over size, shapes and borders and it is not possible to obtain multi-layered particles by ordinary electrospraying techniques.

The advantages of using the technique presented herein, i.e., electrospinning followed by cutting, compared with electrospraying is that it provides full control over final microparticles in term of shape, size, number of layers, possibility of loading a drug substance and degree of compression, thanks to the post-electrospinning cutting procedure leading to formation of particles.

The microparticles may be cut from mono-layered, two-layered, three-layered or multi-layered nanofibrous sheets or a combination thereof.

The microparticles might additionally contain one or more solid layers (e.g., provided with enteric coatings, metal nanoparticles etc.).

The microparticles may also contain one or more active substance(s) per layer. In the case of microparticles cut from a multi-layered nanofibrous material, the one or more active substance may be present in one or more of the multiple layers.

In general, an active substance or any other substance contained in the solution to be electrospun is distributed homogenously in the electrospun sheet obtained. In the case of a multi-layered electrospun sheet, several substances (or excipients) could be distributed through the nanofibers and hence through the whole microparticle.

The microparticles are made from electrospun fibers and comprise one or more polymers. Examples of polymers can be: poly(D,L-lactide-co-glycolide (PLGA), poly-lactic acid (PLA), poly-glycolic acid (PGA), pullulan, silk fibroin, polyvinylpyrrolidone (PVP), methylcellulose, carboxymethylcellulose, ethylcellulose, hydroxypropylcellulose (HPC), hydroxypropyl methylcellulose, acrylates, acrylic acid copolymers, and combinations thereof. It should be mentioned that other materials than polymers can also be mixed with the electrospinning polymers in high weight or volumetric ratios. In the context of the present invention such a material may replace one of the polymers to be used together with one or more polymers.

The microparticles may also contain one or more excipients such as a plasticizer, a release rate modifier, a solubilizer etc. as described herein. Addition of such substances is typically to the solution containing one or more polymer(s) (and, optionally other substances), so that the excipients become part of the nanofibrous structure.

Dependent on the particular use of the microparticles, they may be prepared in a size within the range of 1-500 μm. If the microparticles are intended for nasal delivery, the mean particle size should be in a range of 30 to 120 μm; if the microparticles are for pulmonary delivery the mean particle size is normally in the range of 1 to 20 μm. For other routes such as are buccal, vaginal, rectal, etc. larger sizes are also usable.

Importantly, however, the microparticles should be monodisperse, i.e. the microparticles of the present invention has a very narrow particle size distribution, wherein the size of the particles at the most spans from −10% of the mean particle size to +10% of the mean particle size such as, e.g., ±5% or ±2.5% of the mean particle size.

The nanofibrous sheet(s) (before cutting out the monodisperse microparticles) may have different thicknesses and porosities. In the case of a multi-layered nanofibrous sheet, each individual layer may have a thickness different from or the same as the other layers and the composition of each individual layer may be different or the same. The same applies to porosity and other relevant features. Thus, the final microparticles may be composed of identical or different layers of nanofibrous materials.

The final cutting of the nanofibrous sheet into microparticles may lead to a tunable degree of compactness in the final multilayer nanofibrous microparticle depending on the original thickness of the (e.g., multilayer) electrospun sheet, the depth and sidewall taper angle of the cutting tool, and other factors such a cutting pressure and time.

The electrospun sheet from which the microparticles of the present invention are formed may be subjected to
i) adding an external enlarged surface area by performing post-electrospinning procedures such as electrospraying over the (e.g., multilayer) nanofibrous sheet aiming for enhanced mucoadhesion and increased transit time
ii) loading metallic or magnetic nanoparticles or nanorods such as gold nanoparticles, ZnO nanorods and similar materials for potential tentacle-shaped final microparticles for enhanced muco-adhesion and increased retention
iii) fabricating a stack of various layers (2-10), the various layers may be made from the same polymer or different polymers, and/or from the same active substance or different active substances iv) loading one or more active substance(s) (such as, e.g., drug substance(s)) into nanofibers up to 50% of the total mass thanks to the high loading potential of nanofibers and the potential to perform electrospinning on suspension made from low-soluble drug components.

### Preparation of Monodisperse Microparticles

Described herein is also a method for preparing monodisperse microparticles, the method comprises the steps of
i) electrospinning a solution comprising one or more polymer(s) and, optionally, one or more active substance(s), to obtain a nanofibous sheet comprising the one or more polymer(s) and the optional one or more active substance(s),
ii) cutting the fiber sheet obtained by a cutting tool to obtain monodisperse microparticles with uniform shape and size.

More specifically, a method for preparing monodisperse microparticles comprises the steps of i) electrospinning a solution comprising one or more polymers to obtain a mono-layered nanofibrous sheet or electrospinning several solutions each containing one or more polymer(s), one after another to obtain a multi-layered nanofibrous sheet, the solution(s) may optionally contain an active substance such as a drug substance,
ii) potentially covering the electrospun sheet obtained on top and/or bottom side with a solid polymeric layer of polymer nanoparticles to provide e.g., pH sensitive coatings or propellants e.g., for micromotors,
ii) cutting the nanofibrous sheet using a cutting tool to obtain microparticles with uniform shape and size.

First, one or more polymeric solution(s) (either as single polymer or mixture of polymers) is subjected to electrospinning, which makes a mono-layer from the solution, or layer by layer electrospinning of different polymers on top of each other is done to form a multi-layer. Optionally other ingredients such as one or more active substances such as, e.g, one or more drug substances are also included per electrospinning solution. The resulting product from the electrospinning is a nanofibrous sheet, which is subject to cutting using microfabricated cutting tools having different topographies so that different shapes can be obtained. The cutting results in nanofibrous microparticles having a well-defined shape and having a narrow particle size distribution, i.e., the microparticles are monodisperse microparticles.

The solvent could be water or water-alkanol mixtures, a C₁-C₃ alkanol such as methanol, ethanol, propanol or isopropanol, or mixtures thereof. It may also be a volatile organic solvent such as Chloroform, Dimethylformamide (DMF), Hexafluoroisopropanol (HFIP), Dichloromethane (DCM), Trifluoroacetic acid (TFA).

The choice of solvent depends on the polymer used in the electrospinning process. It is preferred that the polymer is fully dissolved in the solvent to avoid clotting of the syringes of the electrospinning apparatus, and to ensure that a homogenous solution is prepared and subsequently a uniform fiber sheet is obtained.

Almost all conventional polymers applied in biomedical applications have been tried for electrospinning. Except some polymers such as chitosan, which are somehow challenging for electrospinning due to formation of gels in low concentrations; most polymers have shown the potential to make fibers using electrospinning. More common polymers for preparation of the fiber sheet can be listed as poly(D,L-lactide-co-glycolide (PLGA), poly-lactic acid (PLA), poly-glycolic acid (PGA), polyvinyl alcohol (PVA), polyethylene oxide (PEO), pullulan, polyvinylpyrrolidone (PVP), methylcellulose, ethylcellulose, carboxymethylcellulose, hydroxypropylcellulose (HPC), hydroxypropyl methylcellulose, acrylates, acrylic acid copolymers, and combinations thereof.

The fiber sheet is prepared by a method comprising
i) dissolving a polymer in a solvent to obtain a solution,
ii) electrospinning the solution.

If a drug substance is added, the method comprises
i) dissolving a polymer in a solvent to obtain a solution,
ii) adding a drug substance to said solution or dissolving the drug in the relevant solvent to make the drug solution and mixing the drug and the polymer solutions to obtain a resulting mixture
iii) electrospinning the resulting mixture.

If an excipient is added, the method comprises
i) dissolving a polymer in a solvent to obtain a solution,
ii) adding an excipient to said solution to obtain a resulting mixture,
iii) electrospinning the resulting mixture.

If both a drug substance and an excipient are added, the method comprises
i) dissolving a polymer in a solvent to obtain a solution,
ii) adding a drug substance and an excipient to said solution to obtain a resulting mixture,
iii) electrospinning the resulting mixture.

Multiple layers are obtained by repeating the electrospinning process and capturing the new sheet over the sheet already obtained. The layers may be identical, or they may be different. Thus, they contain different polymer(s), and one or more of the layers may contain the same or different actives substance (such as drug substances) or excipients.

The fiber sheet obtained is then cut to the desired shape and size for the microparticles. The cutting is performed by an ultra-sharp cutting tool with cutting edges that penetrate the electrospun fiber sheet smoothly and produce discrete microparticles. The cutting tool features cutting edges where the width or conversely the radius of curvature is kept below 2 μm.

### Monodisperse Microparticles Containing a Drug Substance

The microparticles described herein may be used as a drug delivery system. The drug delivery system may be designed to be administered by oral, nasal, buccal, vaginal, rectal, pulmonal, topic or parenteral administration.

For drug delivery, the microparticles contain a drug substance. The drug substance may be any therapeutically active substance. The drug substance may be electrospun together with the polymeric substance or it may be added to the microparticle obtained, e.g., in connection with formulating the microparticles into a composition such as a pharmaceutical composition.

In general, it is preferred that the electrospinning is performed using a solution of the polymeric substance. The drug substance may be added to this solution and electrospun together with the polymeric substance. The drug substance may be dissolved in the solvent, or it may be present in the polymer solution as discrete particles. In the latter case, the particle size of the drug substance should be kept low (preferably in the range of nanometer, e.g. less than 100 nm) so that it does not clot the syringe of the electrospinning apparatus.

### Excipients

The microparticles described herein may also contain one or more pharmaceutically acceptable excipients. In general, such excipients are electrospun together with the polymeric substance. Such excipients could be added to facilitate the manufacturing process and/or improve the flexibility or processability of the polymer.

Examples of such excipients could be plasticizers such as citric acid esters like acetyl triethyl citrate, tributyl citrate or triethyl citrate, castor oil, diacetylated monoglycerides, dibutyl sebacate, diethyl phthalate, sorbitol, glycerol or glycerol derivatives like triacetin or tributyrin, a cellulose derivative like cellulose nitrate, glycols like polyethylene glycols notably polyethylene glycols with a molecular weight from about 100 to about 1500, polyethylene glycol monomethyl ether, propylene glycol, or mixtures thereof.

Other examples could be excipients functioning as release rate modifiers, i.e., excipients that have impact on the release of a drug substance contained in the microparticle. Normally, plasticizers are also regarded as release rate modifiers and a change in concentration of plasticizer will affect the release rate. Other examples of excipients that may affect the release rate are solubility improving agents that may be added in order to adjust or manipulate the release rate of a drug substance contained in the microparticles. Such a solubility improving agent may be added to the solvent containing the drug substance before being fed into the apparatus making the electrospun sheet of fibers. Examples of solubility improving agents are polyoxyethylene fatty alkyl esters, isopropylester of a straight or branced C₈-C₁₄ fatty acid, a propylene glycol mono- or diester of a C₈-C₁₄ alkanol or alkenol, a straight or branced C₈-C₂₄ alkanol or alkenol, a C₈-C₂₂ acylglyceride, N-alkylpyrrolidone or N-alkylpiperidone, a mineral such as paraffin.

In many cases it is preferred to add one or more pharmaceutically acceptable excipients when formulating the microparticles into a pharmaceutical composition. Such excipients may be selected from the examples mentioned above, and/or they may be pharmaceutically acceptable excipients normally used in formulation of pharmaceutical compositions. The choice of excipients depends on the type of composition. A person skilled in the art can find guidance in Remington's Handbook of Pharmaceutical Excipients. For example, if the composition is for oral administration, it may be in the form of tablets, capsules, sachet etc. and typically excipients are selected from fillers, disintegrants, lubricants, stabilizers, pH-adjusting agents, plasticizers, release rate modifiers, solubilizers etc., whereas if the composition is for administration to the skin or mucosa, it may additionally contain a bioadhesive agent, skin-conditioning agents, anti-irritative agents etc.

### The Microfabricated Cutting Tool

In the present context, a microfabricated tool is used for cutting discrete monodisperse particles out of a premade electrospun sheet consisting of one or more layers. The microfabricated tool can either be composed of isolated compartments or close-packed compartments. In both cases, the compartments are formed by either isolated or interconnected sidewalls that are residing on the top surface of the bulk tool and the sidewalls are terminated by sharp cutting edges as schematically shown in FIG. 2. Alternatively, the microfabricated tool is a mesh/grid in which the grid lines are terminated by sharp cutting edges at the top surface that interfaces the electrospun sheet during cutting. In this case, the gridlines are interconnected (as there is no bulk unstructured volume of the tool) thereby forming a tessellated mesh/grid. In the current invention when a grid-type tool is used, the geometry and spatial size of the compartments or voids determine the geometry and size of the cut microparticles. In the present context, the sidewall taper angle θ is between 45° and 90°. The upper boundary is to ensure that the cut microparticles can be released from the tool and the lower boundary of 45° is to ensure that massive smearing of the nanofibrous morphology of the surface of the electrospun sheet interfacing the cutting tool is avoided. For θ<90° the force applied for cutting is not only focused on the sharp cutting edge but also on the tapered sidewalls which may lead to a potentially undesirable compaction of the cut microparticles and distortion of the nanofibrous morphology. Cutting occurs at the cutting edges that are located on the top of the protrusions or gridlines of the tool. The cutting edge can be a direct continuation of the sidewall in which case the inner taper angle of the cutting edge θᵢ=θ (refer to FIG. 2). Alternatively, the inner taper angle θᵢ<θ which results in a decrease in the width of the protrusion or grid line and ultimately a termination thereof in a sharp cutting edge. In all embodiments of the current invention the cutting edge has a width not exceeding 2 μm. Alternatively, the sharpness of the cutting tool can be quantified in terms of the radius of curvature of the potentially rounded apex constituting the cutting edge. In that case, the radius of curvature is also kept below 2 μm. Especially when cutting small particles for e.g. pulmonary delivery, it is highly attractive to use a tool with very sharp cutting edges in order to avoid a massively compressed flash layer along the periphery of the cut particles. When cutting through electrospun sheets comprising one or more layers, it is essential to also consider the protrusion (i.e., the combined height of the sidewall and the cutting edge) or grid line height. If the overall sheet thickness is larger than the protrusion or grid line height, the sheet will be subject to non-negligible compaction and potential distortion of the nanofibrous morphology in the interface between tool and sheet. If the sheet thickness is smaller than or equal to the protrusion or grid line height, the compaction and morphology smearing in the interface will be reduced, provided that the cutting process parameters are optimized. The cutting tool can either be a solid planar entity or a thin shim that allows for mounting on e.g., a cylindrical roller. In the latter case, the tool material must allow for adaption onto a non-planar geometry (e.g. a cylinder) which prohibits the use of e.g. non-flexible, hard and brittle materials. The microfabricated tool is made in a hard material such as silicon, fused silica, quartz, glass, metal (including alloys), ceramic, organically-modified ceramic (ORMOCER) or certain epoxy-based or polymer materials having sufficient hardness for cutting through the electrospun sheets. In one embodiment of the invention, the tool is made by conventional top-down processing of silicon, fused silica, quartz or glass. This allows for producing tools that are extremely hard but also quite brittle. In another embodiment of the invention, the tool is made in metal or an alloy using conventional processes including micromachining, laser ablation, electric discharge machining, casting or electroplating. Such materials are especially suitable for both solid planar tools as well as for shim-based tools. In yet another embodiment the tool is made by thermal or UV curing of a suitable material such as spin-on-glass, ORMOCER, an epoxy-based material or any other analogue that allows for patterning using a pre-fabricated template. In all embodiments of the present invention, the tool surfaces can be modified by deposition of materials that allow for i) increasing the hardness and durability of the cutting edge and sidewalls (this could e.g., be diamond-like carbon (DLC)) and ii) materials that allow for reducing the adhesion between the tool and the cut microparticles. Such materials could include fluorinated polymers such as perfluorodecyltrichlorosilane (H,1H,2H,2H-perflourodecyltrichlorosilane, FDTS) or other analogues. The hard coating can effectively increase the hardness and toughness of the tool material whereas the anti-stick coating can facilitate release of the microparticles after the cutting process. To facilitate cutting and the subsequent release of the produced microparticles, it is also paramount that the tool surfaces that interface the electrospun sheet during cutting have a low surface roughness. This is especially important when considering the production of very small monodisperse particles in the 1-20 μm range. Therefore, in all embodiments of the current invention, the surface roughness of all surfaces contacting the electrospun sheet is kept below 5 μm. This requirement applies regardless of whether or not hard and/or anti-stick coatings have been deposited on the tool.

### The Cutting Process

The microparticles are produced in a process bearing resemblance to conventional mechanical punching that allows for shaping materials such as rubber or metal into discrete parts using a matching punch and die set mounted on a mechanical, pneumatic or hydraulic press. In the current disclosure, the microscale cutting of electrospun sheets into discrete microparticles is obtained in a room temperature process with a short cycle time. The cutting itself is facilitated by a mechanical, pneumatic or hydraulic force which is applied to the backside of the microfabricated tool. Cutting can be obtained using a planar parallel press or the tool can be mounted on a cylindrical roller, which is then pressed into the electrospun sheet in a continuous roll-to-plate or roll-to-roll process. The force applied to the tool (or conversely to the electrospun sheet) will be focused on the sharp cutting edges residing on the top of the protrusion sidewalls or grid lines. This effectively ensures that the nanofibers are sheared, stretched and broken in the interface between the cutting edge and the sheet. The microscale cutting differs fundamentally from mechanical punching as no counter die is used. Instead, a sheet, plate or film of backing material is situated underneath the electrospun sheet during cutting. It is paramount that the hardness of the backing material is lower than the hardness associated with the top surface of the microfabricated tool, as the cutting edges will penetrate into the backing material during cutting. In addition, the hardness of the tool surface should also exceed the hardness of all materials comprising the electrospun sheet. Cutting occurs when the nanofibrous fracture strain or strains (in the case of a sheet consisting of multiple electrospun layers) is reached. The force needed for obtaining discrete microparticles during cutting depends on the materials comprising the electrospun sheet and the topography of the microfabricated tool. From a production point of view, having a tool with vertical sidewalls (i.e. θ=90°) is beneficial as the pressing force is focused entirely on the cutting edges. As the sidewall taper angle is reduced (i.e. θ<90°), the contact area between tool and sheet is increased continuously as the tool penetrates into the electrospun sheet. Depending on the mechanical properties of the polymeric nanofibers constituting the top layer of the sheet, the increased contact area between tool and electrospun sheet can result in both massive compaction of the microparticles as well as nanofibrous morphology change in or near the interface between tool and electrospun sheet. Said morphology change will most likely be evident as a smearing of the top surface of the produced microparticles. In the case where polymeric materials with glass transition temperatures below room temperature (an example could be polycaprolactone) is used, the cutting process will result in not only morphology changes but also a decidedly polymer flow which ultimately impacts the properties of the microparticles. When using a tool with compartments formed by protrusions featuring sharp cutting edges, the cutting process will be somewhat self-limiting once the electrospun sheet starts interfacing all concave surfaces of the compartments. In this case, the applied cutting force can be translated into a reduced pressure acting on the entire top surface area of the tool. When using a tool consisting of interconnected gridlines where the gridline height is larger or much larger than the electrospun sheet thickness, such a scenario is non-occurring and the tool will continue to penetrate into the backing material until the process is terminated. After cutting, the pressing force is relieved. In general, the micro-cutting process used for making the particles presented in the current disclosure, can be divided into two steps, as schematically shown in FIG. 3, each with a cycle time of approximately 30 s: i) the actual cutting (FIG. 3A), where the sharp edges of the microfabricated tool penetrates through the ES and slightly into the backing material to produce discrete monodisperse microparticles and ii) the transfer of the produced microparticles (FIG. 3B) from the microfabricated tool onto a 65 μm thick plasticized polyvinylchloride (PVC) foil with a 10 μm thick layer of pressure sensitive acrylic-based adhesive (Nitto Semiconductor Wafer Tape SWT 20+R, Nitto Denko Corporation, Osaka, Japan). Two similar stacks are used during cutting and transfer. To protect the lower press plate while simultaneously ensuring a planar and non-rough surface during cut and release, the stacks comprise a 50 μm thick aluminum foil and a φ100 mm single-side polished silicon substrate onto which a flexible PVC foil has been attached (same foil as used during release). As backing material, a high quality aluminum foil (Al foils for thermal imprinting, Obducat Technologies AB, Lund Sweden) with a thickness of approximately 75 μm was used. The Al foil ensures a minimal degree of strain in a plane perpendicular to the applied force during micro-cutting. This is beneficial in terms of minimizing potential deformation of the produced MuNaMi. During the transfer cycle, the backing material was substituted with a 150 μm thick Teflon sheet (Polyfluor Plastics, Minervum, Netherlands) which serves as a flexible anti-stick cushion. The adhesive foil is merely placed on the microstructured surface of the tool and covered by an approximately 100 μm thick polypropylene foil which serves as a protective cushion between the upper press plate and the microfabricated tool.

### General

It should be understood that any feature and/or aspect discussed above in connections with the compounds according to the invention apply by analogy to the methods described herein.

The following figures and examples are provided below to illustrate the present invention. They are intended to be illustrative and are not to be construed as limiting in any way.

### DESCRIPTION OF DRAWINGS

FIG. 1: Schematic demonstration of the potential to cut through a multilayer electrospun sheet, e.g. here 5 layers using a pyramidal frustum cutting tool to get multilayered microparticles.

FIG. 2: Schematics relating to the description of the microfabricated tool. Important features such as the sidewall taper angle θ, the cutting edge type, the sidewalls of the protrusions or grid lines and the overall positioning of the compartments/holes, that dictates the geometry and size of the produced microparticles, have been shown. To the left, 21 is the sidewall of the protrusion or grid line, 22 is the bulk volume of the tool. If a grid is used, 22 is not present and the grid lines are formed by the interconnected sidewalls of the grid lines. If a tool with compartments is used, the protrusions are located on the top surface of the tool and said protrusions are terminated by sharp cutting edges. 23 is the cutting edge and as evident, this apex can have different geometries: the cross section can be triangular but the sidewalls can also be concave rounded. In both cases, the cutting edges are connected to the protrusions or grid lines at the top surface. The sharp cutting edges can also be formed as a direct continuation of the sidewalls or grid lines. The sidewall taper angle θ is indicated in 24 and this angle is in the range from 45-90° in the current disclosure. Note that the sidewall taper angle may be identical to the inner sidewall taper angle θ, associated with the cutting edge, which is shown in 25. 26 and 27 are top-down schematics showing the different types of arrangements of the protrusions/grid lines and the associated cutting edges. As seen in 26, the protrusions or grid lines can either be interconnected and arranged in a regular tessellated pattern or the protrusions (this arrangement is not possible for tools that are microfabricated grids) can be isolated entities arranged in any pattern and having any geometrical shape. In 27, circular protrusions arranged in a regular 2D array can be seen.

FIG. 3: Schematics illustrating the micro-cutting process (FIG. 3A) and the subsequent transfer step (FIG. 3B) where the discrete monodisperse MuNaMi are collected from the microfabricated tool. Two similar stacks are used for micro-cutting and transfer. The aluminum foil, silicon substrate and the flexible PVC foil are used to protect the lower press plate and to ensure a planar surface during micro-cutting. The polymer foil used on the top of the stacks is to protect both the upper press plate as well as the microfabricated tool. The Teflon foil used during release of the MuNaMi is partly to prevent stiction of the mildly adhesive foil to the flexible PVC foil on the silicon substrate. The inserts in the bottom row show a SEM image of pyramidal frustum MuNaMi after release (scale bar corresponds to 200 μm) and a picture of the adhesive foil after release of close-packed pyramidal frustum MuNaMi.

FIG. 4: (A) Silicon tool featuring isolated cylindrical compartments that are formed by protrusions with sharp cutting edges. The tool has four areas of 20×20 mm2, which can each be used for cutting 17689 individual particles in a single cutting cycle. (B) 3D topography data from vertical scanning interferometry measurements on the tool before the final steps associated with the sculpting of the cutting edge. (C) Cross sectional profile from the topography analysis. The inner compartment depth is approximately 55 μm and the diameter at the top surface is approximately 70 μm. (D) Scanning electron microscopy image acquired at a tilt angle of 30°. The image shows the final tool surface featuring isolated compartments with sharp cutting edges. The scale bar corresponds to 200 μm. (E) Top-down scanning electron microscopy image showing the final tool surface. The scale bar corresponds to 300 μm. The inset shows a high magnification image of the sharp cutting edge having a width of approximately 100 nm. The scale bar corresponds to 2 μm.

FIG. 5: (A) Silicon tool featuring close-packed inverse pyramidal frustum compartments that are separated by sharp cutting edges. The overall micropatterned area is 50×50 mm2 and the tool allows for producing 137641 particles in a single cutting cycle. (B) 3D topography data from vertical scanning interferometry measurements on the tool before the final steps associated with the sculpting of the cutting edge. (C) Cross sectional profile from the topography analysis. The compartment depth is approximately 70 μm. (D) Scanning electron microscopy image acquired at a tilt angle of 30°. The Image shows the final tool surface featuring close-packed compartments with sharp cutting edges. The scale bar corresponds to 60 μm. (E) Top-down scanning electron microscopy image showing the final tool surface. The scale bar corresponds to 200 μm. The inset shows a high magnification image of the sharp cutting edge having a width in the sub-100 nm regime. The scale bar corresponds to 2 μm FIG. 6: Representation of the micro-cutting procedure over a monolayer electrospun sheet (PLGA nanofibers) using a microfabricated tool with cylindrical compartments (a) to form a collection of hemisphere-shaped nanofibrous microparticles (b) and a patterned electrospun sheet including micropores (b).

FIG. 7: Schematic representation of multilayer nanofibers (a), the procedure of cutting through a multilayer sheet using sharp cylindrical compartment cutting tool (c), the hemispherical multilayer nanofibrous microparticles (MuNaMi) cut out of the electrospun layer (c), and the zoom-out top view representation of the MuNaMi showing the external fibers (PLGA microfibers shown by arrows) and the internal fibers (pullulan nanofibers shown inside circles).

FIG. 8: Fabrication of whole-layer high yield cut out of pyramidal frustum shaped microparticles (a) using ultra-sharp close packed microfabricated tool (b). A single multilayered microparticle (c) and the collection of particles for storage and future use (d).

FIG. 9: Trying laser ablation to cut microparticles out of pullulan nanofibers. As illustrated in (a-c), it was not possible to cut through nanofibers using different powers and increasing the power resulted in mixed structures (d-f) including empty pores, fused particles and partly cut circles. Further increase of the power resulted in maintaining a microporous patterned sheet as shown in (g-i).

FIG. 10: SEM images of BaSO₄ loaded SilMA-PEO microparticles and their in vivo release study. SEM images of SilMA-PEO microparticles containing different amounts of BaSO₄ (w/w %; 10-50% BaSO₄: SilMA-PEO) are shown (A). Regardless of the amount of loaded BaSO₄, all electrospun layers of BaSO₄-loaded SilMA-PEO could be thoroughly cut to make BaSO₄ loaded SilMA-PEO nanofibrous microparticles. A pilot in vivo study on rats for oral delibery of BaSO₄-Loaded SilMA-PEO microparticles demonstrated that these particles were detectable in both stomach and intestine. There was an accumulation of particles in stomach along with more individual particles inside small intestine and colon three hours after dosing the capsule (B).

FIG. 11: The sustained tunable release of AMX from the multilayer microparticles and the appearance of PLGA-AMX:pullulan-PLGA MuNaMi inside the capsule used for release study.

FIG. 12: Comparing the release between three constructs of AMX:pullulan monolayer sheet, PLGA-AMX:pullulan-PLGA multilayer sheet, and the microparticles cut out of the PLGA-AMX:pullulan-PLGA multilayer sheet, and their schematic representations.

FIG. 13: The overall appearance of frustum pyramid-shaped PLGA-AMX:pullulan-PLGA microparticles (a) and the difference between external part (b) and the internal part (c) where the whole external part is made by the thicker PLGA fibers, whilst the internal part consists of three sublayers including 1) the PLGA fibres with larger diameter originally making the top layer, 2) pullulan fibers with smaller diameter originally making the internal layer, and 3) the PLGA fibres with larger diameter originally making the bottom layer in the multilayer sheet undergoing the cutting procedure.

FIG. 14: Demonstrating how the original thickness of the electrospun sheet (Hₑₛ) compared with the depth of cutting tool (Hp) affect the final 3D structure and compactness of the final microparticles. Schematic demonstration of dimensional changes from an electrospun sheet (A) to nanofibrous microparticles (B) and the relevance to the different original thicknesses of the electrospun sheet (C), where final constructs demonstrate increased compactness and sharpness of edges along with increasing the original thickness of sheets (D).

FIG. 15: Quantitative comparison of the dimension of the base square or circle in the frustum pyramid (A) or hemispherical (B) nanofibrous microparticles respectively. As demonstrated almost identical monodisperse particles with small variations from the averages are fabricated,

FIG. 16: An example of MoNaMi made out of silk fibroin nanofibers demonstrating the rows of monodisperse fabricated particles (a), an individual particle (b), and the ribbon-shaped nanofibers making the electrospun sheet.

FIG. 17: An example of MoNaMi made out of Eudragit S100 nanofibers demonstrating the monodisperse fabricated particles (a), and an individual particle (b),

FIG. 18: An in vivo study comparing three different structures (a) in terms of the absorption (b) and presence of a small molecule (referred to as compound x) inside gastrointestinal tract (c,d). As illustrated in (b) both the pH-responsive construct and MoNaMi have reduced absorption of compound X in the small intestine and thus lower Cmax values in plasma. Also, the behaviour of these constructs is different in terms of intestinal content (FIG. 18 (b),(c)), where MoNaMi shows much faster transition compared with the two sheet structures.

### EXAMPLES

### Example 1—the Journey to Manage High Yield Monodisperse Monolayer Nanofibrous Microparticles

To benefit from both nano- and micro-structures for smart drug delivery applications, the inventors aimed at cutting microparticles out of electrospun nanofibrous sheets. The inventors started with applying laser ablation on electrospun sheets of different thicknesses and different polymers such as PCL, PLGA and pullulan. Preliminary investigations revolving around using laser micromachining for producing microparticles from electrospun sheets comprising one or more layers, were conducted on a dedicated laser micromachining tool (microSTRUCT™ vario, 3D-Micromac AG, Chemnitz, Germany). We investigated laser ablation of electrospun sheets having an approximate thickness of 80 μm. Laser ablation was conducted using a 50 W picosecond laser (FUEGO, Time-Bandwith (now Lumentum), San Jose, CA, USA) emitting at 1064 nm. The focal length of the lens was 250 mm.

After performing some optimizations, we concluded that the focal diameter of ablation on electrospun samples is larger than the ablation spot sizes, and led to complications for making microparticles.

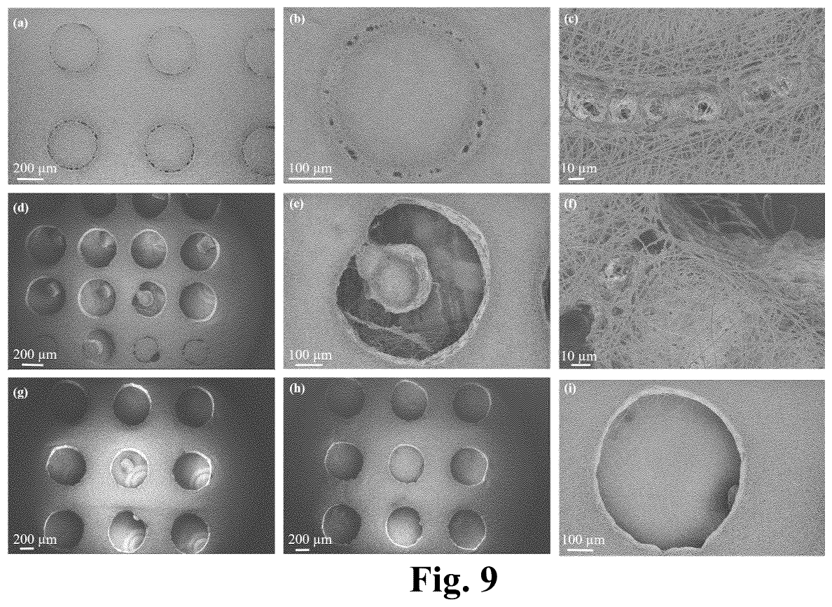

For instance, increasing the laser power resulted in transitions from superficial cuts through the electrospun layer where in many cases a thin uncut residual layer was present after laser micromachining (FIG. 9 (a)-(c)), to mixture of different structures including fused particles (FIG. 9 (d)-(f)), or to fully burnt particles leaving a microporous nanofibrous layer behind (FIG. 9 (g)-(i)). Moreover, we often observed large deviations between the design files used for cutting and the actual particle size—mostly due to unwanted ablation and local melting. Ultimately, such complications terminated the work on laser cutting electrospun microparticles at an early stage. We concluded that laser ablation is not an appropriate tool for making microparticles covering the full range from 1-500 μm. Looking for a more robust and reproducible technique which requires no heat or wet processing for making microparticles out of drug-loaded nanofibers, we considered to apply a custom-made microfabricated tool to cut through the nanofibers, where pressure was the driving force to cut through the nanofiberous sheet. The first version of our applied cutters was prepared as normal Si cutting tools. Having low yields and incomplete cutting through the electrospun layer, we further fabricated ultra-sharp cutting tools as described in examples 2 and 3.

### Example 2—Microfabricated Tool with Near-Vertical Sidewalls and Isolated Cutting Edges

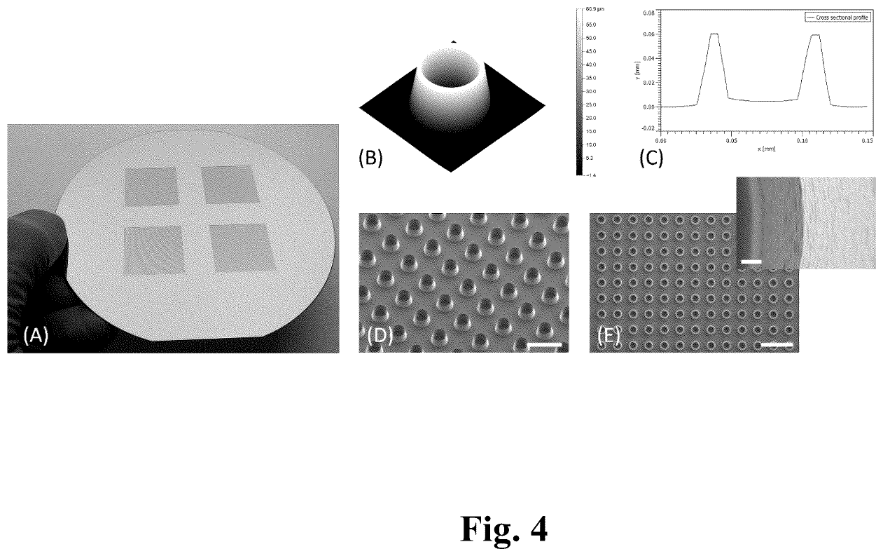

In order to demonstrate effective cutting of electrospun sheets using a tool with near-vertical sidewalls and isolated protrusions with ultra-sharp cutting edges, a suitable tool was fabricated by conventional top-down processing of silicon substrates (525 μm thickness, single-side polished, <100>, Topsil GlobalWafers A/S). Initially a 1.1 μm thick wet thermal SiO₂ layer was grown in a dedicated oxidation furnace (Tempress horizontal furnace). The oxide layer is used for defining a hard mask used in the subsequent dry etching steps. The oxide layer was masked using UV lithography (MLA100 Tabletop Maskless Aligner, Heidelberg Instruments) in a 1.5 μm thick layer of the positive resist AZ©5214E (MicroChemicals GmbH, Ulm, Germany). The resist was exposed with a dose of 90 mJ/cm² @365 nm and developed in AZ© 726 MIF (MicroChemicals GmbH, Ulm, Germany) for 90 s using a single-puddle approach (SOss MicroTec Gamma 2M developer). The resist pattern was then transferred into the SiO₂ layer using an Advanced Oxide Etcher (AOE, STS MESC Multiplex ICP) with 5 sccm C₄F and 174 sccm H₂ as the reactive gasses. The etch was conducted using a chamber pressure of 4.0 mTorr, a coil power of 1300 W and a platen power of 200 W. This effectively results in an oxide hard mask that can be used for etching deep into the silicon substrate. Prior to the silicon etching steps, the resist was stripped using a combination of energetic oxygen plasma in a barrel asher (300 Semi Auto Plasma Processor, PVA TePla America Inc.) and submersion into 7-up at 80° C. The 7-up consists of concentrated H₂SO₄ with (NH₄)₂S₂O₈ salt added just prior to immersion into the solution, that effectively strips remaining traces of resist. The first step in the etching process is a Si taper etch performed in an Advanced Silicon Etcher (ASE, STS MESC Multiplex ICP). The etch, that creates a slightly tapered sidewall profile, uses SF₆ (75 sccm) and O₂ (60 sccm) with a trace amount of C₄F (5 sccm) added for etching into the silicon. The etch was carried out in continuous mode using a chamber pressure of 40 mTorr, a platen temperature of 20° C., a coil power of 2000 W and a platen power of 15 W to ensure a modest mask undercut in combination with a relatively low surface roughness. The cylindrical protrusions that constitute the sharp cutting edges, were etched to a depth of approximately 55 μm and the remaining SiO₂ mask was stripped in buffered hydrofluoric acid (12 vol % HF with NH₄F). Prior to the oxide removal, the wafers were cleaned using oxygen plasma and a mixture of concentrated H₂SO₄ and H₂O₂ (i.e., Piranha, H₂SO₄:H₂O₂=4:1). The taper profile was subsequently subject to a dry isotropic etch in the ASE which was performed to reduce surface roughness and ultimately to increase the sharpness of the cutting edge. The isotropic etch was merely implemented by omitting the passivation step in the Bosch process which is used for silicon deep etch. The reactive gasses were SF₆ (230 sccm) and O₂ (23 sccm) and the substrate temperature was kept at 20° C. The manual pressure setting was 87.7% and the coil power and platen power was 2800 W and 19 W, respectively. As the fabricated tool was used for efficiently cutting through nanofibrous electrospun sheets, the silicon substrate was cleaned using oxygen plasma prior to immersion in piranha solution and deposition of an anti-stick coating. This coating was deposited using molecular vapor deposition (MVD 100 Molecular Vapor Deposition System, Applied Microstructures Inc.) where a conformal monolayer of 1 H,1 H,2H,2H-perflourodecyltrichlorosilane (FDTS) is formed on the exposed silicon surface. The decrease in surface energy, which results from the FDTS deposition, ensure that nanofibrous micro-particles can easily be detached from the Si protrusions after pressure assisted cutting. Each silicon wafer has four individual 20×20 mm² tools that each support cutting of 17689 identical particles in a single short-cycle-time process (FIG. 4). Based on inspection using optical microscopy (Nikon ECLIPSE L200), vertical scanning interferometry (PLu Neox 3D Optical Profiler, Sensofar Metrology, Terrassa, Spain) and scanning electron microscopy (SEM, Zeiss Supra 40 VP) the diameter of the cylindrical protrusions at the cutting edge was close to 70 μm and the average height of the protrusions was 55 μm. The associated taper angle of the protrusions was approximately 82⁰ and the cutting edge line width was in the sub-100 nm regime.

### Example 3—Microfabricated Tool with Sloped Sidewalls and Close-Packed Cutting Edges

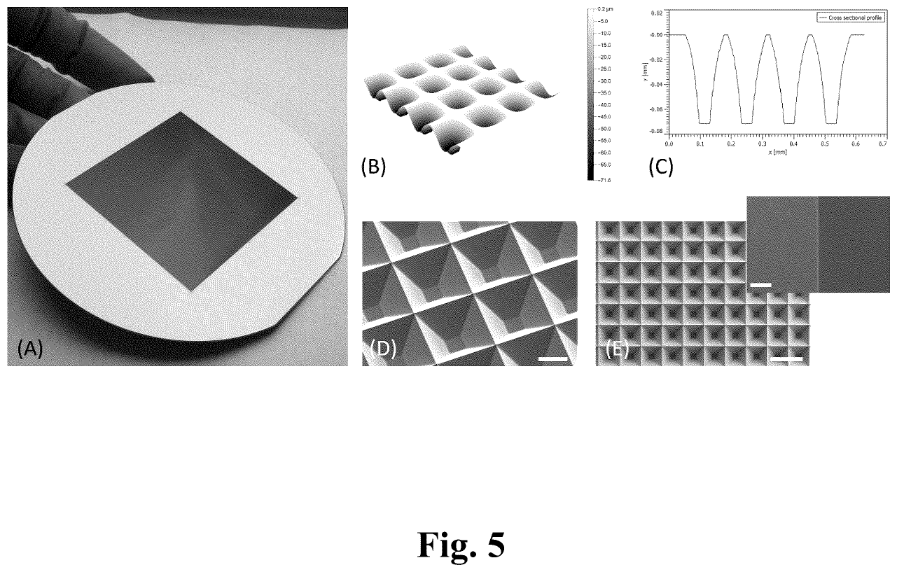

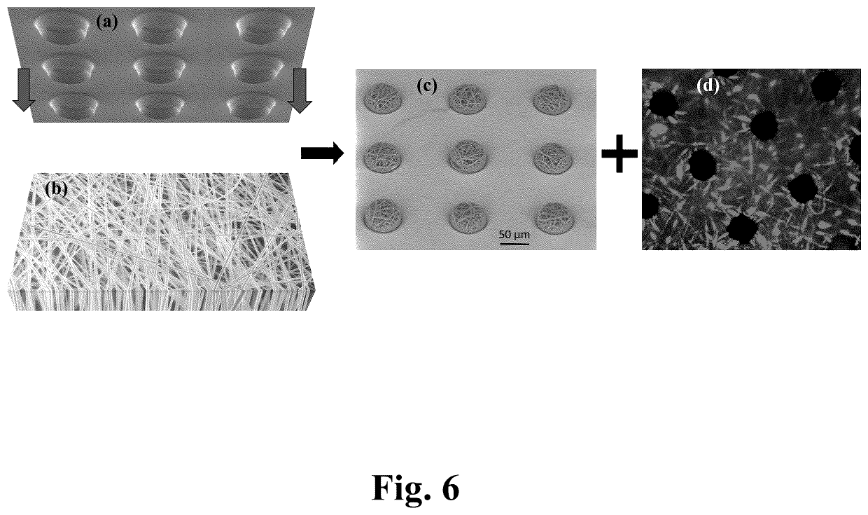

In order to increase the number of particles produced per cutting cycle and to demonstrate the feasibility of the cutting method when using a tool with moderately tapered sidewalls a close-packed silicon tool featuring interconnected cutting edges for making particles in the shape of regular pyramidal frustums was produced (FIG. 5). To this end, conventional top-down processing of silicon (525 μm thickness, single-side polished, <100>, Topsil GlobalWafers A/S) was applied. As in the previous example, a 1.1 μm thick wet thermal SiO₂ layer was grown in a dedicated oxidation furnace (Tempress horizontal furnace). Subsequently, the oxide layer which will constitute the hard mask during etching, was selectively removed using a combination of UV lithography (MLA150 WM I, Maskless Aligner, Heidelberg Instruments) in 1.5 μm thick positive resist AZ©5214 E (MicroChemicals GmbH, Ulm, Germany) and Advanced Oxide Etching (AOE, STS MESC Multiplex ICP). In the UV lithography step, automatic flat alignment was employed, and a dose of 75 mJ/cm² @375 nm was used before developing the exposed resist for 90 s in AZ© 726 MIF (MicroChemicals GmbH, Ulm, Germany) using a single-puddle approach (SOss MicroTec Gamma 2M developer). The resist pattern was then transferred into the SiO₂ layer using a process with 5 sccm C₄F₈, 4 sccm H₂ and 174 sccm He as the reactive gasses. The etch was conducted using a chamber pressure of 4.0 mTorr, a coil power of 1300 W and a platen power of 200 W. After etching, the remaining resist mask was removed using a combination of energetic oxygen plasma in a barrel asher (300 Semi Auto Plasma Processor, PVA TePla America Inc.) and submersion into 7-up at 80° C. The 7-up consists of concentrated H₂SO₄ with (NH₄)₂S₂O₈ salt added just prior to immersion into the solution, that effectively strips remaining traces of resist. To achieve moderately tapered sidewalls, wet anisotropic etching of silicon was used as the first step. The SiO₂ masked substrate was immersed in 28 wt % KOH heated to 80° C. to ramp up the silicon etching rate. As the anisotropic KOH etch features major differences in the etch rates along different crystal orientations, holes having an inverse pyramidal frustum shape were produced in the unmasked portions of the silicon substrate. Before the final etching step, the remaining SiO₂ hard mask was stripped in buffered hydrofluoric acid (12 vol % HF with NH₄F) and the substrate was subject to a 10 min clean at 80° C. in concentrated H₂SO₄ with (NH₄)₂S₂O₈ salt added just prior to immersion. This step removes potential traces of contaminants on the substrate surface. In order to sculpt the local topography associated with the cutting edges, a brief isotropic silicon dry etching step was employed on an Advaned Silicon Etcher (ASE, STS MESC Multiplex ICP). The isotropic etch was implemented by omitting the passivation step in the Bosch process which is used for silicon deep etch. The reactive gasses were SF₆ (230 sccm) and O₂ (23 sccm) and the substrate temperature was kept at 20° C. The manual pressure setting was 87.7% and the coil power and platen power was 2800 W and 19 W, respectively. As in the previous example, the top surface of the silicon tool was coated with a thin anti-stick layer (MVD 100 Molecular Vapor Deposition System, Applied Microstructures Inc.) constituted by a conformal monolayer of 1H,1H,2H,2H-perflourodecyltrichlorosilane (FDTS). Based on inspection using optical microscopy (Nikon ECLIPSE L200), vertical scanning interferometry (PLu Neox 3D Optical Profiler, Sensofar Metrology, Terrassa, Spain) and scanning electron microscopy (SEM, Zeiss Supra 40 VP), the tool was characterized post production. The produced silicon tool features a 50×50 mm² central region harboring 137641 inverted pyramidal frustum compartments that are close-packed and where the sharp cutting edges are interconnected. The distance between neighboring cutting edges is 135 μm, the depth of the compartments is approximately 70 μm and the side length associated with the bottom surface of the inverted pyramidal frustum is approximately 32 μm. The aforementioned topographical information allows for calculating an approximate sidewall taper angle of 53.7° and based on high resolution SEM inspection, the broadness of the cutting edges is in the sub-100 nm regime.

### Example 4: Monolayer Nanofibrous Microparticles (Hemishpercial MoNaMi)

To try the microfabricated cutting tools (FIGS. 4 and 5), the inventors started with cutting through a monolayer electrospun sheet. In order to fabricate a monolayer electrospun sheet, polymers such as pullulan, SilMA-PEO, and PLGA were used. PLGA (poly lactic-co-glycolic acid) with a lactide:glycolide ratio 50:50 and molecular weight of Mw=54,000-69,000 Da was purchased from Sigma-Aldrich Denmark. pullulan (molecular weight~200 000 Da) was kindly donated by Hayashibara Co., Ltd. (Okayama, Japan). Bombyx Mori silk cocoons was purchased from Wild Fibers, UK. Sodium carbonate (Na2CO3), Lithium bromide (LiBr), Glycidyl methacrylate (GMA) solution (211.5 mM), Dimethyl formamide (DMF), chloroform, and all other reagents were the analytical grades and purchased from Sigma-Aldrich Denmark. Water was purified using a Milli-QPlus 185 water purification system (Millipore, Bedford, MA) with resistivity higher than 18 MQ-cm. Amoxicillin Trihydrate (AMX) with the purity of >98% was purchased from TCI chemicals. For synthesis of methacrylated silk (SilMA), we started with extraction of silk fibroin (SF) first. To degum silkworm cocoon, the exterior sericin protein was removed by boiling silkworm cocoon (40 g; each silkworm cocoon was sliced into 4 pieces) in 1 L of 0.05 M Na2CO3 solution for 30 min at 100° C. Then the silk fibroin filaments were washed with distilled water for several times and were dried at room temperature. Subsequently 20 g of degummed silk was dissolved in 100 mL of 9.3 M lithium bromide (LiBr) solution at 60° C. for 1 h. Having SF solved by LiBr, 3 mL of GMA solution (Sigma-Aldrich, Denmark) was slowly dropped to the mixture stirring with a speed of 300 rpm for 3 h at 60° C. to create a high yield reaction between GMA and SF. Then, the resulting solution was filtered through a miracloth (Calbiochem, San Diego, USA) and dialyzed against distilled water using 12-14 kDa cutoff dialysis tubes for 4 days. After dialysis, the SilMA solution was purified by centrifugation (3000 rpm, 10 min, 4° C.) to remove the undissolved impurities. Finally, the concentration of the SilMA solution was calculated and was stored at 4° C. for further use. For electrospinning of monolayer sheet of the selected polymer(s), the respective solutions were electrospun onto stainless steel static collector using a home-made electrospinning setup. For all polymers (pullulan, SilMA-PEO and PLGA), polymer solutions were loaded into 1-mL syringes fitted with a 21-gauge blunt-end needle, and the electrospinning parameters were set at an applied voltage of 12-14 kV, a collection distance (grounded collector from needle tip) of 15 cm, and the electrospinning solution flow rate of 0.5 (SilMA-PEO) or 1.0 mL/h (others). Electrospinning was performed at room temperature (25° C.) and at a relative humidity of 35%. For electrospinning of PLGA, PLGA was dissolved in Chloroform/DMF (90:10 v/v) with a concentration of 20% (w/v). For pullulan electrospinning, pullulan was dissolved in water with concentration of 20% w/v. For electrospinning of SilMA, aqueous SilMA solution of around 6% (w/v) was mixed manually with PEO solution of 6% (w/v) in the ratio of 3:1 (v/v) and the mixture underwent electrospinning. PEO was added to assist with electrospinning of SilMA. An example of cutting microparticles out of a monolayer electrospun sheet of PLGA is shown in FIG. 6. For cutting, first, a cylindrical micro-cutter (FIG. 6(a)) was pressed over the mono-layer electrspun PLGA (FIG. 6(b)), where hemi-spherical microparticles are cut through the electrospun layer (FIG. 6(c)); and the remaining part (microporous nanofibrous layer; FIG. 6(d)) can be further used as a patterned electrospun sheet. We further tested the feasibility of cutting other electrospun mono-layer sheets such as pullulan, and SilMA-PEO. All resulted in around 100% yield and full-thickness cut-out of the fibrous electrospun sheets.

The average diameter of the fibers constituting the electrospun sheets and their morphology were analyzed using Scanning electron microscopy (SEM). Samples were mounted on stubs and were coated with a thin layer of platinum to increase conductivity. Imaging was performed using a Zeiss Supra 40 VP scanning electron microscope. Fiber diameters were determined using ImageJ with a minimum of 100 measurements per sample.

### Example 6—Multilayer Nanofibrous Microparticles (Hemispherical and Pyramid Shaped MuNaMi)

With increasing attention towards the use of multilayer nanofibers for potential loading and delivery of different drugs/bioactive agents, a three-layered sheet of PLGA-pullulan-PLGA was also electrospun using the same setup mentioned above. First, 0.25 mL of PLGA was electrospun using a solution of 20% PLGA in mixed solvent of chloroform/DMF (90:10 v/v), then a layer of pullulan 20% was added (1 mL), and finally the last layer of PLGA 20% was electrospun on top of the two other layers (0.25 mL).

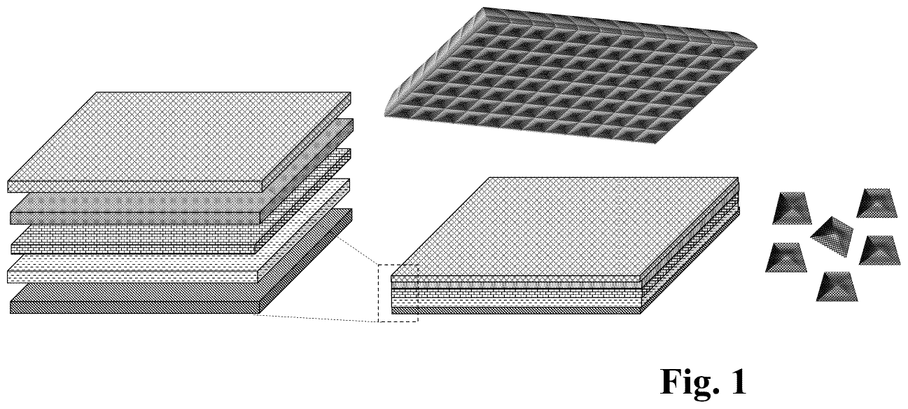

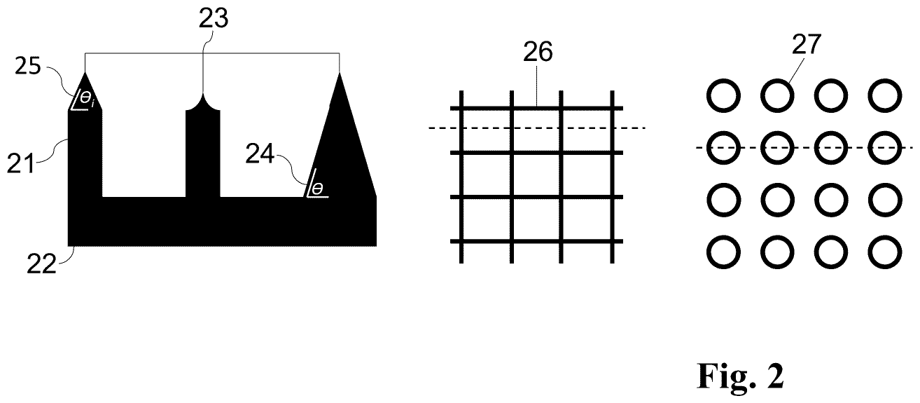

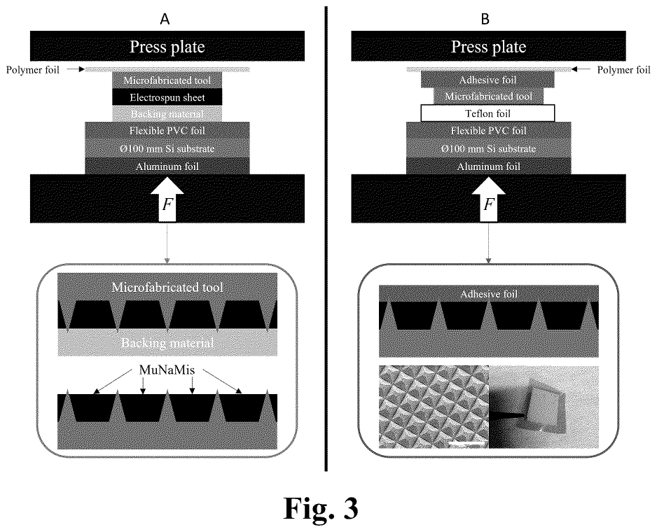

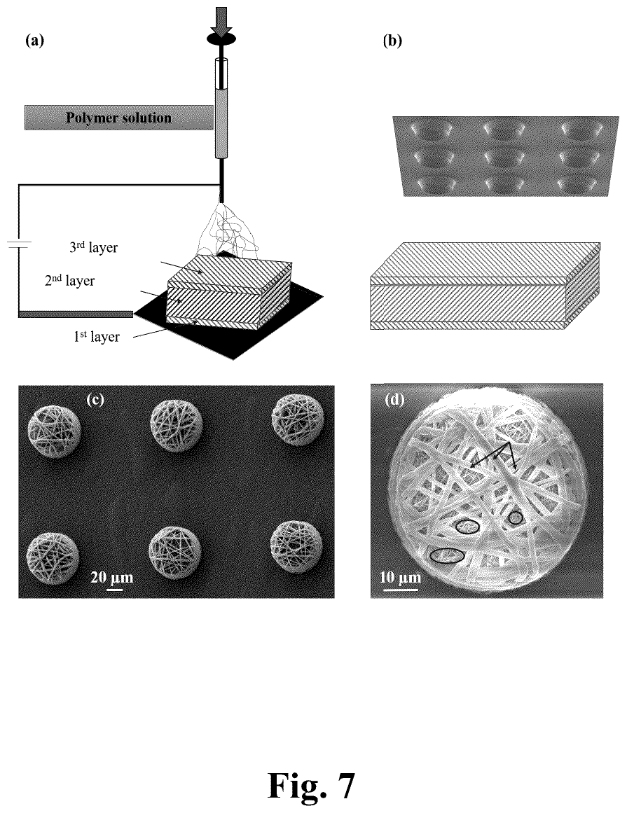

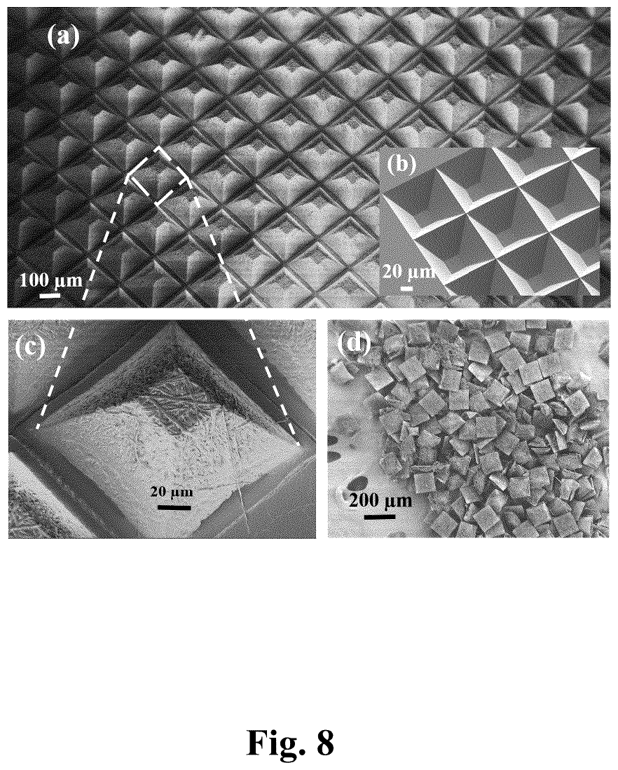

Then, we used the ultra-sharp Si cylindrical cutter to cut through the multilayer sheet of PLGA-pullulan-PLGA (FIG. 7). FIG. 7(a) shows a schematic representation of electrospinning for making a multilayer electrospun sheet, where applying the cutting tool (FIG. 7(b)) leads to formation of multilayered nanofibrous microparticles with uniform size and shapes (FIG. 7(c)) with detectable layers due to the different average diameters of polymeric nanofibers in the multilayered sheet (FIG. 7(d)). The sheet shown in FIG. 7 is made by a layer-by-layer electrospinning of PLGA, pullulan and PLGA respectively to fabricate a three-layer sheet of PLGA-pullulan-PLGA with an approximate thickness of 10-80-10 μm and average nanofiber diameters of 785±423 nm and 282±76 nm for PLGA and pullulan fibrous layers respectively. Pressing the ultra-sharp Si tool on the layered sheet (FIG. 7(b)) forms multilayered microparticles (FIG. 7c), where the external layers (PLGA fibers shown by arrows in FIG. 7(d)) extends over the marginal area of the internal layer (pullulan nanofibers shown inside the circles in FIG. 7(d)) from both sides and cover the whole internal layer. In the next step, we fabricated pyramidal frustum shaped microparticles (FIG. 8). Aiming for increased output from micro-cutting where the whole electrospun sheet can be cut into microparticles (FIG. 8(a)), we applied the ultrasharp cutting tool (FIG. 8(b)), leading to 100% yield along with 100% cut-out of the original multilayer PLGA-pullulan-PLGA electrospun sheet (FIG. 8(c)), where particles can easily get collected (FIG. 8(d)). In FIG. 1, the potential to increase the number of layers in the multilayer sheet (here 5 layers) has been schematically outlined. Making different layers using a combination of selected hydrophilic and/or hydrophobic polymers is an advantage to provide the possibility of loading various types of drugs depending on their solubility, bioavailability and the expected release trend. In general, layered sheets are favorable for sustained release applications. For instance, Milosevic et al.⁸ applied a computational model to validate a prolonged drug release from a three-layered fibrous scaffold (PCL/PLGA/PCL) compared with the monolayer PLGA scaffold. They demonstrated that the three-layered scaffold delays the drug release process and can be used in postoperative therapy for the time-controlled release of the drugs. Likewise, multilayered microparticles could be superior for tunable release not only based on number of layers, types of polymers and the loaded drug, but also depending on the thickness of the layers and the degree of coverage provided by the external layers entrapping the internal content. We have applied PLGA and pullulan to have a combination of hydrophobic-hydrophilic biopolymers to make a three-layered sheet. PLGA is widely applied as a microcarrier in the therapeutic formulations thanks to its biocompatibility and biodegradability, as well as the potential for tuning a sustained release of encapsulated hydrophobic drugs. On the other side, the hydrophilic pullulan is an edible ideal polymer to encapsulate hydrophilic drugs or bioactive agents with poor stability.

### Example 7—BaSO₄-Loaded SilMA-PEO Nanofibrous Microparticles for Tracing the Microparticles Through Gut

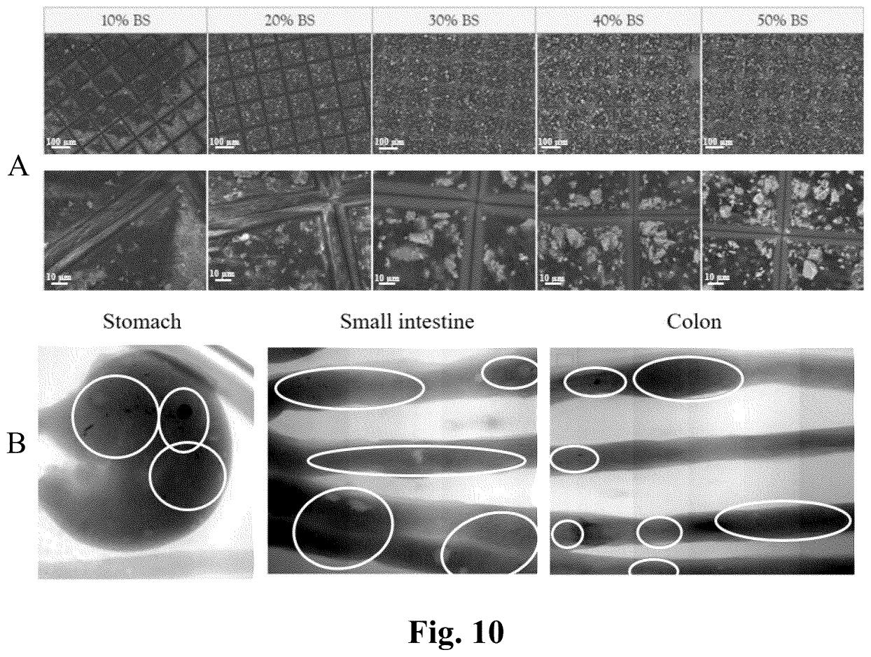

BaSO₄-SilMA nanofibers were fabricated using different ratios of BaSO₄ to SilMA (w/w; 10, 20, 30, 40, 50%) and were cut successfully using an ultra-sharp cutting tool (FIG. 10A). A pilot preliminary in vivo study on 6 rats was performed where the microparticles were put inside a capsule (no enteric coating) and the capsule was fed to rats. The whole gastro-intestinal tract was removed after pre-defined time-points of 1 hour or 3 hours and was imaged using X-ray imaging to study if microparticles were detectable, how the distribution was and what the retention time of particles would be. It was shown (FIG. 10B) that BaSO₄-Loaded SilMA-PEO microparticles were released in both stomach and intestine. Although there was an accumulation of particles in stomach, individual particles were also detectable inside small intestine and colon 3 hours after dosing the capsule.

### Example 8—PLGA-Pullulan-PLGA MuNaMi for Sustained Delivery of AMX

Amoxicillin (AMX) was used as a drug model in this study. AMX was loaded into pullulan nanofibers with concentration of 5% (w/w; weight of AMX to the weight of polymer) making either a monolayer AMX:pullulan or a multilayer electrospun sheet of PLGA-AMX:pullulan-PLGA where AMX:pullulan is the internal layer. For encapsulation of AMX in pullulan, AMX was dissolved in water and then pullulan powder was added to the solution to make the desired concentration. For making 5% AMX to pullulan, the 5% AMX to pullulan solution was made using a 1% aqueous suspension of AMX. It is worth nothing that the maximum solubility of AMX (the tri-hydrate form) in water is around 0.4% (or 4 mg/mL), and therefore, a suspension of AMX was used for making 5% AMX to pullulan nanofibers. The multi-layer PLGA-AMX:pullulan-PLGA sheet underwent cutting as well using cutting tool with a close-packed inverse pyramidal frustum topography. Finally the AMX:pullulan monolayer sheet, the PLGA-AMX:pullulan-PLGA multilayer sheet and the PLGA-AMX:pullulan-PLGA multilayer microparticles were used for drug delivery test. For release study on amoxicillin from microparticles, microparticles were loaded into gelatin capsules (Torpac size 9). The capsules were coated in a 12% (w/v) solution of Eudragit L100 in IPA where dibutyl sebacate was added as plasticizer in a 5% w/w ratio relative to Eudragit. At the beginning of the experiment, one capsule was inserted in a cellulose membrane dialysis tubing (MW cut off 12000 Da), the ends were sealed, and it was placed in a beaker including 5 mL of 0.02 M HCl solution with pH 1.7 simulating the acidic conditions present in the stomach. 200 μL samples were taken after 5, 15, 30 and 60 min and the corresponding volume was replaced each time with 0.02 M HCl solution. After taking the last sample, the dialysis bags were transferred in 5 mL phosphate buffer with pH 7.5 simulating the transition to the intestinal environment. 200 μL samples were taken after 5, 10, 20, 30, 30, 45, 60, 90, 120, 240, 360 min and 24 h and the corresponding volume was replaced each time with phosphate buffer. All the taken samples were frozen at −18° C. for HPLC analysis. HPLC analyses were performed using a reverse-phase C₁₈ column high performance liquid chromatography (HPLC). Amoxicillin was analyzed using HPLC in isocratic mode and mobile phases were constituted of A) 6.8 g/L of potassium phosphate monobasic in deionized water and B) deionized water with 5% v/v acetonitrile. The ratio of the mobile phases A:B was 95:5 v/v. Samples were run using a Luna 5 μm C18 100 Å, 250×4.6 mm column (Phenomenex ApS, Nordic Region, Vorlose, Denmark) at room temperature. The injected volume was 20 μL with a flow rate of 1 mL/min and a total run time per sample of 10 min. The wavelength selected for measuring absorbance was 230 nm. Two calibration curves were prepared using a 0.5 mg/mL amoxicillin stock solution dissolved in either 0.02 HCl solution or in phosphate buffer. The calibration range chosen was between 0.1 μg/mL and 100 μg/mL. Prior to analyses, samples were thawed at room temperature and well mixed.

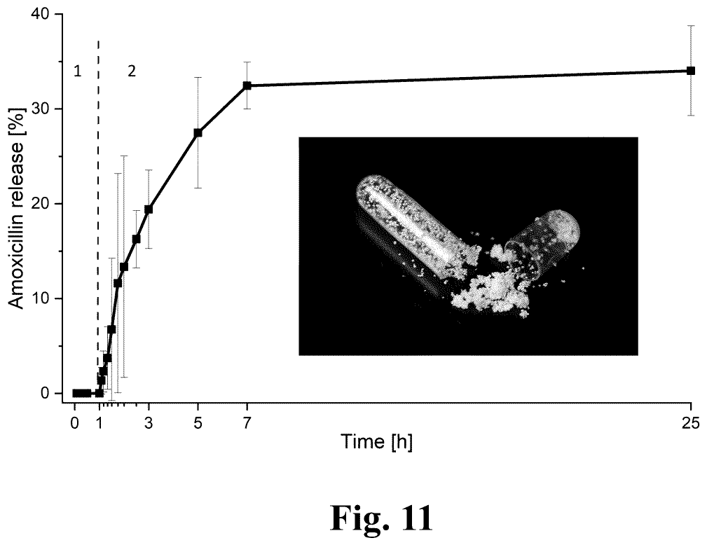

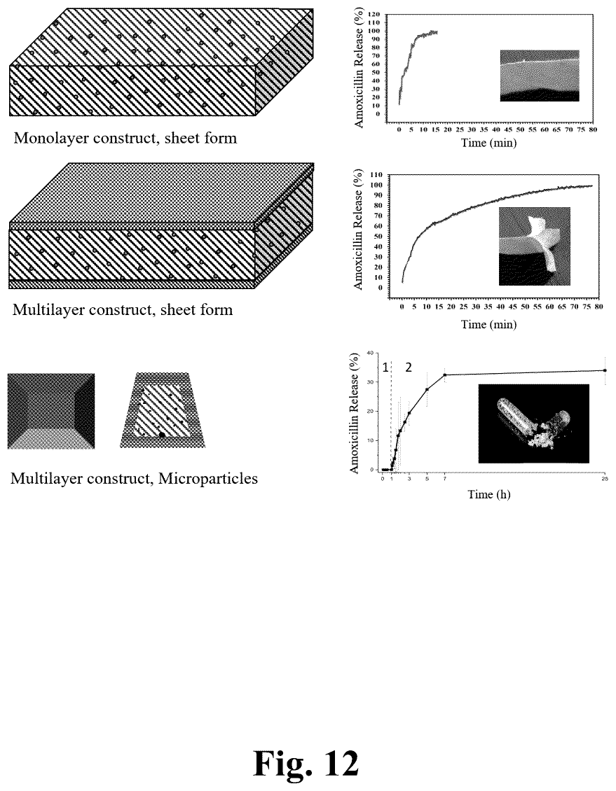

The release result shown in FIG. 11, demonstrates that there has been no release of AMX under acidic conditions (stomach-like environment) during the first one hour, but a sustained release was observed under pH 7.5 simulating the transition to the intestinal environment. The average release after 24 hours under pH 7.5 is less than 35%, representing the sustained release of AMX from the hydrophilic fast soluble pullulan layer. To get a better idea about the effect of forming microparticles out of nanofibers on drug delivery, we compared the release of AMX (pH 7.5) between a monolayer sheet (AMX:pullulan), a multilayer sheet (PLGA-AMX:pullulan-PLGA) and multilayered microparticles which were cut out of the same multilayer sheet (FIG. 12). As shown in FIG. 12, there is a burst release from monolayer sheet of AMX:pullulan (less than 10 min), and an almost burst release from multilayer sheet (80 min). However, the cutting procedure has led to a sustained release, where only 35% of drug is released after 24 hour. The sustained release is usually the preferred form of release where introducing an applied drug to the body. The drug delivery shown in FIG. 12 is primarily high enough to give a rapid onset of the desired therapeutic response. Then the trend maintains a constant level for the desired duration of time.

### Example 9—MuNaMi Constructs Demonstrate Extra Protection for Internal Layer

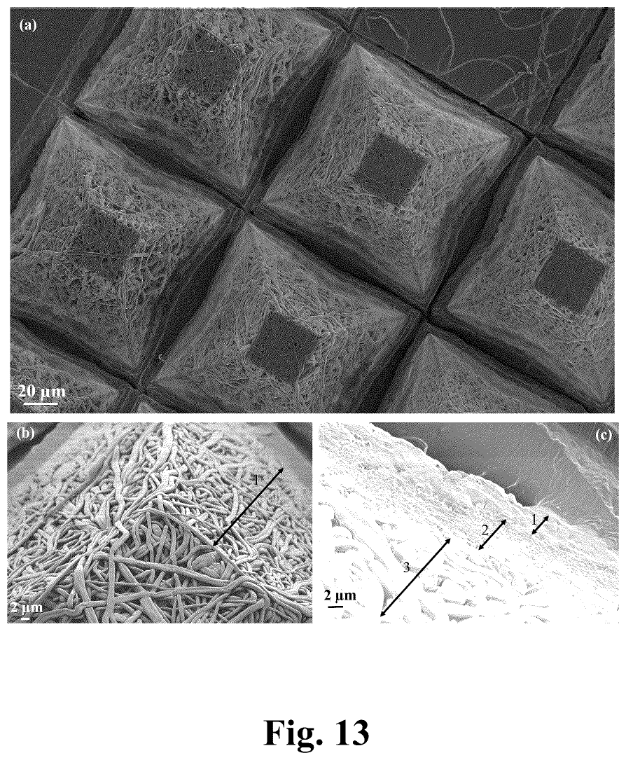

As mentioned in Example 8, applying the originally same PLGA-AMX:pullulan-PLGA multilayers in the two forms of flat sheets (FIG. 12, middle row), or the microparticles cut out of the flat sheets (FIG. 12, bottom row) led to a much more sustained release. It happens due to the role of extra coverage introduced by extension of the surrounding PLGA layer around the pullulan layer during cutting as shown in FIG. 13. As illustrated in FIGS. 13 (a) and (b) the multilayered microparticles are covered by external PLGA layers fully on the external side, where the internal layer (pullulan) is sandwiched between the external layers in the internal view (FIG. 13(c)). This sandwiching can explain the reason for the sustained release of the AMX from the pullulan internal layer.

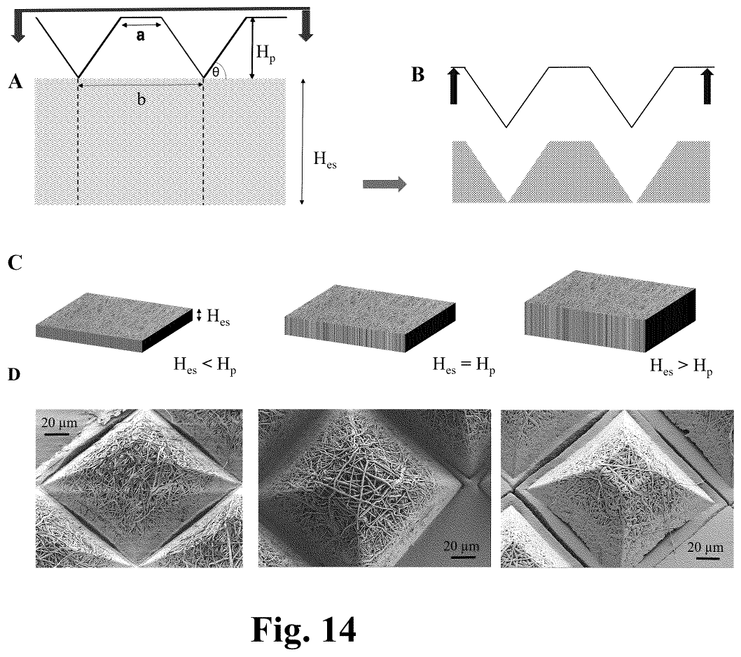

The other explanation that can be considered for the transition from fast to sustained release after cutting the multilayer electrospun sheet to MuNaMi is the effect of compression (FIG. 14) leading to more dense structures wherein the release of trapped drug is postponed resulting in more sustained release. We further investigated how the initial thickness (height) of the electrospun sheet, compared to the depth of the compartments in the cutting tool, would affect the final 3D structure and compactness of fibers in the MuNaMi. During micro-cutting, the electrospun sheet will be compressed (FIG. 14 A,B) and due to volume conservation the level of increased compactness can be evaluated based on geometrical parameters pertaining to the electrospun sheet and the topography/geometry of the micro-cutting tool. The volume of the original electrospun sheet which is cut to form a single particle is calculated according to Eq. 1, where b is the length of the base part (corresponding to the cutting edges) of the concave compartment and Hₑₛ is the thickness or height of the original electrospun sheet. The length of the upper part of the pyramidal frustum, a, is calculated to 27 μm, using the data of the cutting tool as mentioned earlier and according to Eq. 2.

The volume of the particle is considered the same as the cutting tool compartment according to Eq. 3 which is calculated to Vₚ=4.9·10⁻⁴ mm³. Three different PLGA electrospun sheets with thicknesses of around 150, 80 and 60 μm were used as the substrate for micro-cutting (FIG. 14 C,D). As shown in FIG. 14D, a sharp pyramidal frustum shape is obtained when cutting through the thicker sheet of 150 μm, whilst cutting through the other two leads to lower compression, less compactness, and more smooth structures. For the case of using electrospun sheet with original thickness of Hₑₛ=150 μm, the Vₑₛ is calculated to Vₑₛ=2.5·10⁻³ mm³, and therefore the compactness ratio is around 80% according to Eq. 4. Likewise, the compactness of the other two electrospun sheets of PLGA with Hₑₛ of 80 and 60 μm (FIG. 14D) is calculated to 64% and 51% respectively. Therefore, starting with ES of different thicknesses compared with the cutting tool is a scalable possibility to form particles of different compactness and tunable release profile.

$$Volume of the ES making one particle : {V}_{es} = ( {H}_{es} {b}^{2} ) \qquad Eq . 1$$

$$Length of the square in the top part of the particle : a = b - 2 ( \frac{{H}_{p}}{tan θ} ) \qquad Eq . 2$$

$$Volume of frustum pyramid particle : {V}_{p} = \frac{1}{3} {H}_{p} ( {a}^{2} + ab + {b}^{2} ) \qquad Eq . 3$$

$$Compactness : C = ( 1 - \frac{{V}_{p}}{{V}_{es}} ) \cdot 100 \qquad Eq . 4$$

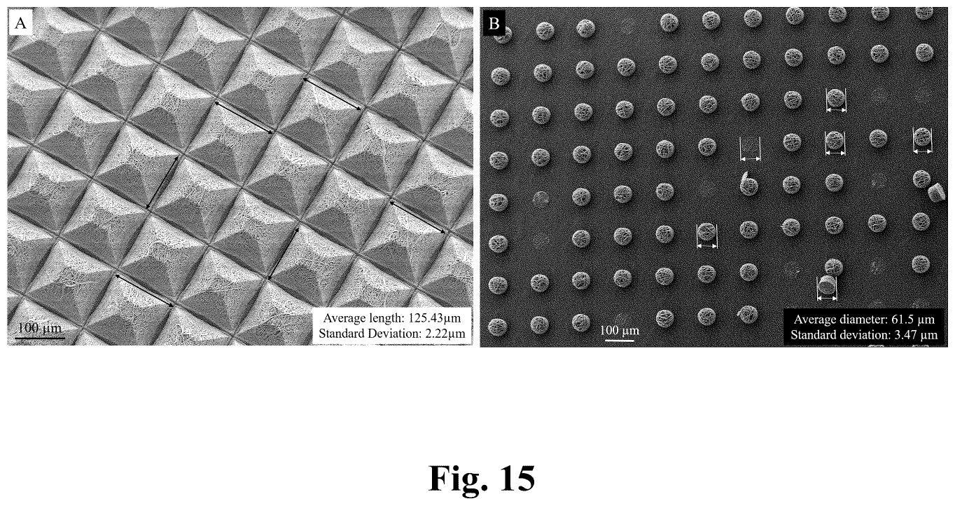

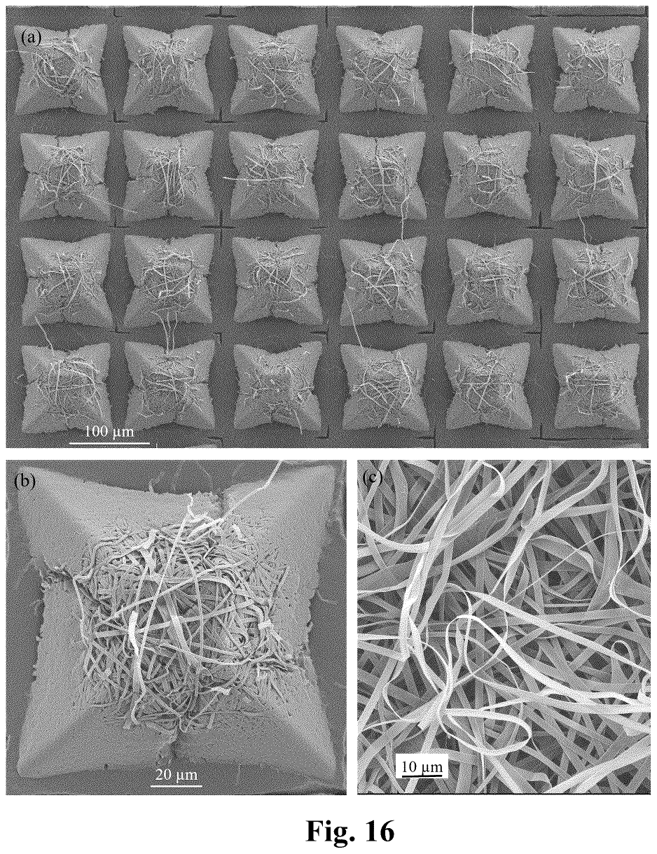

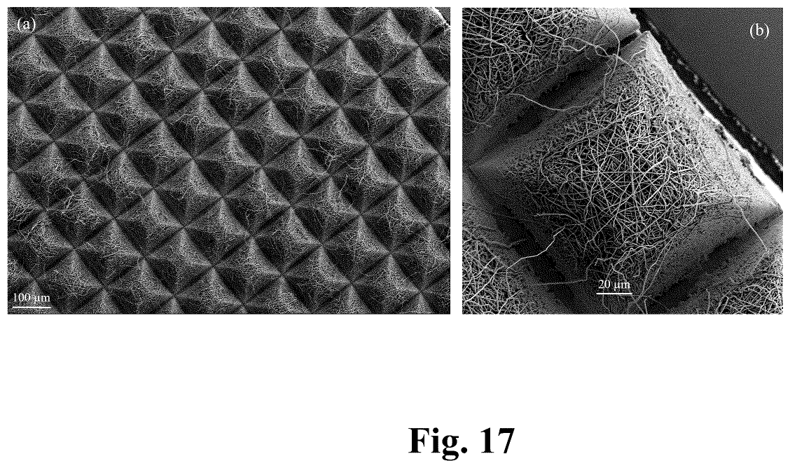

Example 10: For certain applications of the microparticles presented in the current disclosure, it is important to ensure that the microparticles are monodisperse. By using the micro-cutting method outlined herein, it is possible to obtain bulk amounts of microparticles which exhibit very limited variations in terms of the overall geometry and size. This is readily evident from FIG. 6 (c), FIG. 7 (c), FIG. 8, FIG. 13 (a), FIG. 16 (a) and FIG. 17 (a). To substantiate that monodisperse MoNaMis and MuNaMis can indeed be obtained we have further evaluated the size in a more quantitative manner by image analysis of SEM images acquired after release of the produced microparticles. The SEM images, shown in FIG. 15, show ensembles (>30 particles) of microparticles having either a pyramidal frustum (FIG. 15 A) or a hemispherical (FIG. 15 B) geometry. We have used characteristic length scales (the major side length of the pyramidal frustums and the diameter of the hemispherical particles) to quantitatively evaluate the monodispersity by direct measurements of the average side length and diameter and the associated standard deviations. For the pyramidal frustum particles in FIG. 15 A, the average side length was 125.43 μm±2.22 μm and for the hemispherical particles in FIG. 15 B, the average diameter was 61.5 μm±3.47 μm. This effectively translates to size variations below 2% for the pyramidal frustum particles and approximately 5% for the hemispherical particles. It is important to underline that the overall geometric shape of the produced microparticles is largely governed by the geometry of the concave compartments associated with the micro-cutting tool.

However, the exact size (as described by a characteristic length scale or the volume) is influenced in a non-trivial manner by the composition and thickness of the electrospun sheet and factors (e.g. cutting force/pressure and mechanical properties of the backing layer) associated with the micro-cutting itself. This entails that the exact size may differ from the size of the concave compartments of the cutting tool in a non-negligible manner. Nonetheless, it should be clearly evident from the figures in the current disclosure and this example that the particles are indeed subject to minimal variations in overall geometrical shape and size.

### Example 11: To Demonstrate the Potential to Cut Electrospun Sheets Out of Different Polymers, we

have added two extra examples of cutting through monolayer electrospun sheet of silk fibroin (FIG. 16) and Eudragit S100 (FIG. 17) as a pH-dependant polymer. In both cases, monodisperse fully cut particles of the same shape and structure are formed. Despite the different overall shapes of particles obtained from silk fibroin (star-shaped particles in FIGS. 16 (a) and (b) based on the ribbon-shaped silk fibroin fibers) and Eudragit S100 (free-flying fibers due to the fluffy electrospun sheet of Eudragit S100 (FIGS. 17 (a) and (b)), the relevant particles are following almost same structures and dimensions confirming the formation of monodisperse particles.

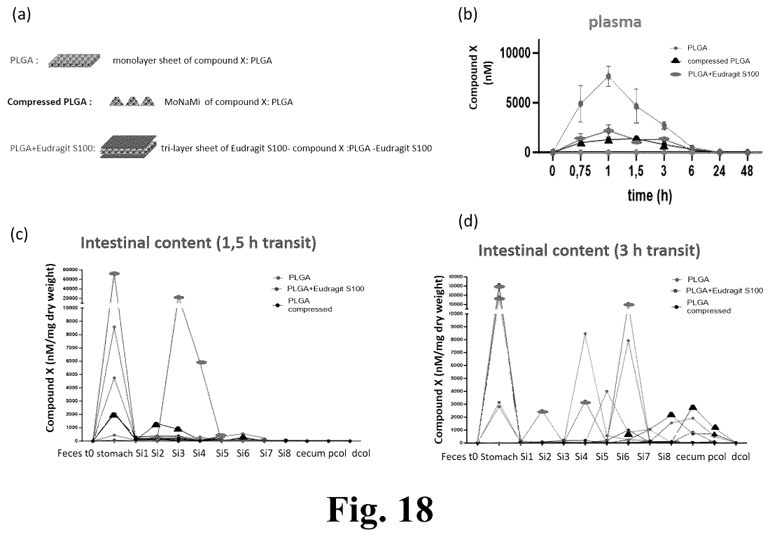

Example 12: We performed a pilot in vivo study on germ-free mice to compare three different structures (FIG. 18 (a)) in terms of the absorption (b) and presence of a small molecule (referred to as compound x) inside the gastrointestinal tract (FIGS. 18 (c) and (d)). The three different structures fabricated to load a small molecule into 1) monolayer electrospun sheet of PLGA (PLGA), tri-layer pH-responsive electrospun sheet of Eudragit S100—PLGA: compound X—Eudragit S100 (PLGA+Eudragit), and the MoNaMi cut-out microparticles out of PLGA: compound X (compressed PLGA). As illustrated in FIG. 18 (b) both the pH-responsive construct and MoNaMi have reduced absorption of compound X in the small intestine and thus lower Cmax values in plasma. It could be related to the protection from the pH-responsive Eudragit in the tri-layer construct and the delayed released from MoNaMi due to the compactness introduced after cutting. Interestingly, the behaviour of these two constructs is different in terms of intestinal content, where MoNaMi shows much faster transition (reaching colon in 3 hours) compared with the two sheet structures (still in stomach and small intestine). Such differences can give different applications and eliminate the need to use extra pH-responsive layers for protection from e.g. stomach acid.

### REFERENCES

1. Augustine, R. et al. Nanoparticle-in-microparticle oral drug delivery system of a clinically relevant darunavir/ritonavir antiretroviral combination. Acta Biomater. 74, 344-359 (2018).
2. Spindler, L. M. et al. Nano-in-Micro-Particles Consisting of PLGA Nanoparticles Embedded in Chitosan Microparticles via Spray-Drying Enhances Their Uptake in the Olfactory Mucosa. Front. Pharmacol. 12, 2282 (2021).
3. Ruggeri, M. et al. Smart nano-in-microparticles to tackle bacterial infections in skin tissue engineering. Mater. Today Bio 16, (2022).
4. Boda, S. K., Chen, S., Chu, K., Kim, H. J. & Xie, J. Electrospraying Electrospun Nanofiber Segments into Injectable Microspheres for Potential Cell Delivery. ACS Appl. Mater. Interfaces 10, 25069-25079 (2018).
5. John, J. V. et al. Engineering Biomimetic Nanofiber Microspheres with Tailored Size, Predesigned Structure, and Desired Composition via Gas Bubble-Mediated Coaxial Electrospray. Smal/16, (2020).
6. Yan, S. et al. Biomimetic Natural Silk Nanofibrous Microspheres for Multifunctional Biomedical Applications. ACS Nano 16, 15115-15123 (2022).
7. Park, C. G. et al. Nanostructured mucoadhesive microparticles for enhanced preocular retention. Acta Biomater. 10, 77-86 (2014).
8. Milosevic, M. et al. Preparation and modeling of three-layered PCL/PLGA/PCL fibrous scaffolds for prolonged drug release. Sci. Rep. 10, (2020).

1. Monodisperse microparticles having a uniform shape, wherein the microparticles comprise electrospun fibers.

2-19. (canceled)

20. The microparticles according to claim 1, wherein the microparticles are in the form of mono-layered or multi-layered fibers.

21. The microparticles according to claim 1 further comprising an active substance.

22. The microparticles according to claim 1, wherein the electrospun fibers comprise an active substance.

23. The microparticles according to claim 21, wherein the active substance is a drug substance.

24. The microparticles according to claim 1 comprising one or more polymers selected from the group consisting of: poly(D,L-lactide-co-glycolide (PLGA), poly-lactic acid (PLA), poly-glycolic acid (PGA), pullulan, polyvinylpyrrolidone (PVP), methylcellulose, carboxymethylcellulose, ethylcellulose, hydroxypropylcellulose (HPC), hydroxypropyl methylcellulose, acrylates, acrylic acid copolymers, and combinations thereof.

25. A method for delivering an active substance to a subject comprising administering the microparticles of claim 21 to a subject.

26. The method according to claim 25, wherein the active substance is a drug substance.

27. The method according to claim 26, wherein the microparticles are delivered by oral, nasal, pulmonal, topic or parenteral administration.

28. The microparticles of claim 1, wherein the particle size distribution spans from −10% of the mean particle size to +10% of the mean particle size.

29. The microparticles of claim 1, wherein the mean particle size is 1 μm to 30 μm or 30 μm to 120 μm.

30. A composition comprising microparticles as defined in claim 1.

31. The composition according to claim 30 further comprising a pharmaceutically acceptable excipient.

32. The composition according to claim 30, further comprising a drug substance.

33. The composition according to claim 30, wherein the composition is formulated for oral, nasal, pulmonal, topic or parenteral administration.

34. A method for preparing microparticles as defined in claim 1, the method comprising:
i) electrospinning a liquid comprising one or more polymers and, optionally, one or more active substances, to obtain a fiber sheet comprising the one or more polymers and the optional one or more active substances, and
ii) cutting the fiber sheet obtained by a cutting tool to obtain monodisperse microparticles with uniform shape and size.

35. The method according to claim 34, wherein the one or more polymers is dissolved or suspended in a solvent.

36. The method according to claim 34, wherein the cutting tool has a single sharp apex.
